(19) United States
(12) Patent Application Publication
(10) Pub. No.: US 2010/0112063 A1
(43) Pub. Date: May 6, 2010

(54) METHOD FOR PREPARING A HYDROGEL ADHESIVE HAVING EXTENDED GELATION TIME AND DECREASED DEGRADATION TIME

(76) Inventors: Garret D. Figuly, Wilmington, DE (US); Samuel David Arthur, Wilmington, DE (US); Robert Ray Burch, Exton, PA (US); Helen S.M. Lu, Wallingford, PA (US)

Correspondence Address: E I DU PONT DE NEMOURS AND COMPANY;LEGAL PATENT RECORDS CENTER, BARLEY MILL PLAZA 25/1122B, 4417 LANCASTER PIKE, WILMINGTON, DE, 19805, US

(21) Appl. No.: 12/145,737

(22) Filed: Jun. 25, 2008

Related U.S. Application Data
(60) Provisional application No. 60/937,593, filed on Jun. 28, 2007.

(51) Int. Cl. A61K 9/10; A61K 31/715; A61K 31/717; A61K 31/718; A61K 31/765; A61P 41/00

(52) U.S. Cl. 424/486; 424/488; 514/54; 514/57; 514/60; 514/59; 424/78.37

(57) ABSTRACT

A method for extending the gelation time of an oxidized polysaccharide to react with a water-dispersible, multi-arm amine to form a hydrogel is disclosed. The extension of the gelation time is accomplished by using a chemical additive. The method also extends the time for the hydrogel to become tack-free, and may also be used to decrease the degradation time of the hydrogel. The chemical additive reacts with the functional groups of the oxidized polysaccharide or the water-dispersible, multi-arm amine, thereby reducing the number of groups available for crosslinking. The use of the resulting hydrogel for medical and veterinary applications is described.

### CROSS-REFERENCE TO RELATED APPLICATION

This application claims priority under 35 U.S.C. §119 from U.S. Provisional Application Ser. No. 60/937,593, filed Jun. 28, 2007.

### FIELD OF THE INVENTION

The invention relates to the field of medical adhesives. More specifically, the invention relates to a method for extending the gelation time of an oxidized polysaccharide to react with a water-dispersible, multi-arm amine to form a hydrogel. The method also extends the time for the hydrogel to become tack-free, and may also be used to decrease the degradation time of the hydrogel.

### BACKGROUND OF THE INVENTION

Tissue adhesives have many potential medical applications, including wound closure, supplementing or replacing sutures or staples in internal surgical procedures, adhesion of synthetic onlays or inlays to the cornea, drug delivery devices, and as anti-adhesion barriers to prevent post-surgical adhesions. Conventional tissue adhesives are generally not suitable for a wide range of adhesive applications. For example, cyanoacrylate-based adhesives have been used for topical wound closure, but the release of toxic degradation products limits their use for internal applications. Fibrin-based adhesives are slow curing, have poor mechanical strength, and pose a risk of viral infection. Additionally, fibrin-based adhesives do not covalently bind to the underlying tissue.

Several types of hydrogel tissue adhesives have been developed, which have improved adhesive and cohesive properties and are nontoxic (see for example Sehl et al., U.S. Patent Application Publication No. 2003/0119985, and Goldmann, U.S. Patent Application Publication No. 2005/0002893). These hydrogels are generally formed by reacting a component having nucleophilic groups with a component having electrophilic groups, which are capable of reacting with the nucleophilic groups of the first component, to form a crosslinked network via covalent bonding. However, these hydrogels typically swell or dissolve away too quickly, or lack sufficient adhesion or mechanical strength, thereby decreasing their effectiveness as surgical adhesives.

Kodokian et al. (copending and commonly owned U.S. Patent Application Publication No. 2006/0078536) describe hydrogel tissue adhesives formed by reacting an oxidized polysaccharide with a water-dispersible, multi-arm polyether amine. These adhesives provide improved adhesion and cohesion properties, crosslink readily at body temperature, maintain dimensional stability initially, do not degrade rapidly, and are nontoxic to cells and non-inflammatory to tissue. However, the gelation time of the hydrogel tissue adhesive is quite rapid, typically less than 10 seconds. For certain applications, a slower gelation would be desirable. For example, in an intestinal anastomosis procedure the mixed adhesive components should gel slowly enough to allow the mixture to be applied around the entire circumference of the intestine and form a complete seal. If the mixture of components gels too quickly, the entire anastomosis site may not be sealed properly due to poor application, clogging of the applicator, or failure of the adhesive to bond to itself once it cures. Additionally, slower gelation would be desirable for use in minimally invasive surgeries, such as laparoscopic surgery, where the mixture of components is delivered by means of a long tube. In other applications, a decreased degradation time may be desirable. For example, for adhesion prevention the hydrogel adhesive should not persist at the site once the healing process has begun.

Therefore, the problem to be solved is to provide a hydrogel tissue adhesive material having a gelation time and a degradation time that can be easily modulated.

Applicants have addressed the stated problem by discovering a method for extending the gelation time and decreasing the degradation time of a hydrogel formed by reacting an oxidized polysaccharide with a multi-arm amine using certain chemical additives.

### SUMMARY OF THE INVENTION

In various embodiments, the invention provides methods for extending the gelation time and decreasing the degradation time of a hydrogel formed by reacting an oxidized polysaccharide with a multi-arm amine using certain chemical additives. The chemical additive reacts with the functional groups of the oxidized polysaccharide or the water-dispersible, multi-arm amine, thereby reducing the number of groups available for crosslinking.

Accordingly, in one embodiment the invention provides a method for extending the gelation time for at least one oxidized polysaccharide (component A) and at least one water-dispersible, multi-arm amine (component B) to form a hydrogel in an aqueous medium, said at least one oxidized polysaccharide containing aldehyde groups, having a weight-average molecular weight of about 1,000 to about 1,000,000 Daltons and an equivalent weight per aldehyde group of about 90 to about 1500 Daltons, and said at least one water-dispersible, multi-arm amine having at least three of its arms terminated by a primary amine group, and a number-average molecular weight of about 450 to about 200,000 Daltons; said method comprising:

contacting component A and component B in the presence of an aqueous medium and at least one chemical additive to form a mixture that forms a resulting hydrogel, wherein said chemical additive is biocompatible, has a molecular weight of less than about 2,000 Daltons and comprises at least one reactive group capable of reacting with amine or aldehyde groups, said reactive group being selected from the group consisting of aldehyde, ketone, glyoxal, acetoacetate, activated ester, imidoester, maleimide, p-nitrophenyl ester, activated halide, anhydride, carbonyl imidazole, epoxide, alkylhalide, H⁺, OH⁻, primary amine, secondary amine, and carboxyhydrazide, provided that the chemical additive does not induce gelation when mixed in the aqueous medium with either component (A) alone or component (B) alone;

wherein, in said method, the additive is used in an amount sufficient to extend the gelation time of components (A) and (B) under predetermined conditions by at least about 10% compared to that of said components (A) and (B) under said conditions, but in the absence of said additive.

In another embodiment, the invention provides method for decreasing the degradation time of a hydrogel formed from at least one oxidized polysaccharide (component A) and at least one water-dispersible, multi-arm amine (component B) in an aqueous medium, said at least one oxidized polysaccharide containing aldehyde groups, having a weight-average molecular weight of about 1,000 to about 1,000,000 Daltons and an equivalent weight per aldehyde group of about 90 to about 1500 Daltons, and said at least one water-dispersible, multi-arm amine having at least three of its arms terminated by a primary amine group, and a number-average molecular weight of about 450 to about 200,000 Daltons; said method comprising:

contacting component A and component B in the presence of an aqueous medium and at least one chemical additive to form a mixture that forms a resulting hydrogel, wherein said chemical additive is biocompatible, has a molecular weight of less than about 2,000 Daltons and comprises at least one reactive group capable of reacting with amine or aldehyde groups, said reactive group being selected from the group consisting of aldehyde, ketone, glyoxal, acetoacetate, activated ester, imidoester, maleimide, p-nitrophenyl ester, activated halide, anhydride, carbonyl imidazole, epoxide, alkylhalide, H⁺, OH⁻, primary amine, secondary amine, and carboxyhydrazide, provided that the chemical additive does not induce gelation when mixed in the aqueous medium with either component (A) alone or component (B) alone;

wherein, in said method, the additive is used in an amount sufficient to decrease the degradation time of the resulting hydrogel under predetermined conditions by at least about 10% compared to that of the hydrogel formed under said conditions, but in the absence of said additive.

In another embodiment, the invention provides a method for forming a hydrogel on an anatomical site on tissue of a living organism by either

(a) mixing on said anatomical site in the presence of an aqueous medium at least one oxidized polysaccharide containing aldehyde groups, having a weight-average molecular weight of about 1,000 to about 1,000,000 Daltons and an equivalent weight per aldehyde group of about 90 to about 1500 Daltons, and at least one water-dispersible, multi-arm amine wherein at least three of its arms are terminated by a primary amine group, wherein the multi-arm amine has a number-average molecular weight of about 450 to about 200,000 Daltons, to form a mixture that forms a hydrogel with a determinable gelation time and a determinable degradation time, or

(b) mixing said at least one oxidized polysaccharide and said at least one multi-arm amine in the presence of an aqueous medium to form said mixture and applying said mixture to said anatomical site to form said hydrogel thereon with said determinable gelation time and said determinable degradation time, the improvement comprising the step of:

including in said mixture at least one chemical additive, wherein said chemical additive is biocompatible, has a molecular weight of less than about 2,000 Daltons and comprises at least one reactive group capable of reacting with amine or aldehyde groups selected from the group consisting of aldehyde, ketone, glyoxal, acetoacetate, activated ester, imidoester, maleimide, p-nitrophenyl ester, activated halide, anhydride, carbonyl imidazole, epoxide, alkylhalide, H⁺, OH⁻, primary amine, secondary amine, and carboxyhydrazide, provided that the chemical additive does not induce gelation when mixed in the aqueous medium with either said at least one oxidized polysaccharide alone or said at least one multi-arm amine alone, whereby the resulting mixture forms a resulting hydrogel;

wherein said additive is used in an amount sufficient to (i) increase the determinable gelation time by at least about 10%; (ii) decrease the determinable degradation time by at least about 10%; or (iii) both (i) and (ii).

### SEQUENCE DESCRIPTIONS

The invention can be more fully understood from the following detailed description and the accompanying sequence descriptions, which form a part of this application.

The following sequences conform with 37 C.F.R. 1.821-1.825 (“Requirements for Patent Applications Containing Nucleotide Sequences and/or Amino Acid Sequence Disclosures—the Sequence Rules”) and are consistent with World Intellectual Property Organization (WIPO) Standard ST.25 (1998) and the sequence listing requirements of the EPO and PCT (Rules 5.2 and 49.5(a-bis), and Section 208 and Annex C of the Administrative Instructions). The symbols and format used for nucleotide and amino acid sequence data comply with the rules set forth in 37 C.F.R. §1.822.

SEQ ID NO:1 is the amino acid sequence of the peptide used as a chemical additive to extend the gelation time of a hydrogel as described in Example 12.

### DETAILED DESCRIPTION OF THE INVENTION

Disclosed herein are methods for extending the gelation time and decreasing the degradation time of a hydrogel tissue adhesive formed by reacting an oxidized polysaccharide with a multi-arm amine. The methods make use of certain chemical additives which comprise at least one reactive group capable of reacting with amine or aldehyde groups. The chemical additive reacts with either the aldehyde groups of the oxidized polysaccharide or the amine groups of the multi-arm amine, thereby reducing the number of functional groups available for crosslinking to form the hydrogel, resulting in an extended gelation time.

The resulting hydrogel is useful as an adhesive for medical and veterinary applications including, but not limited to, wound closure, supplementing or replacing sutures or staples in internal surgical procedures such as intestinal anastomosis and vascular anastomosis, tissue repair, ophthalmic procedures, and minimally invasive surgeries (e.g., laparoscopic surgery). Additionally, the polymer adhesive may have utility in drug delivery, and in anti-adhesive applications.

The following definitions are used herein and should be referred to for interpretation of the claims and the specification.

The term “oxidized polysaccharide” refers to a polysaccharide which has been reacted with an oxidizing agent to introduce aldehyde groups into the molecule.

The term “equivalent weight per aldehyde group” refers to the weight-average molecular weight of the oxidized polysaccharide divided by the number of aldehyde groups introduced in the molecule.

The term “water-dispersible, multi-arm amine” refers to a polymer having three or more polymer chains (“arms”), which may be linear or branched, emanating from a central structure, which may be a single atom, a core molecule, or a polymer backbone, wherein at least three of the branches (“arms”) are terminated by a primary amine group. The water-dispersible, multi-arm amine is water soluble or is able to be dispersed in water to form a colloidal suspension capable of reacting with a second reactant in aqueous solution or dispersion.

The term “water-dispersible, multi-arm polyether amine” refers to a branched polyether, wherein at least three of the branches (“arms”) are terminated by a primary amine group, which is water soluble or able to be dispersed in water to form a colloidal suspension capable of reacting with a second reactant in aqueous solution or dispersion.

The term “polyether” refers to a polymer having the repeat unit [—O—R]—, wherein R is a hydrocarbylene group having 2 to 5 carbon atoms.

The term “branched polyether” refers to a polyether having one or more branch points (“arms”), including star, dendritic, comb, highly branched, and hyperbranched polyethers.

The term “dendritic polyether” refers to a highly branched polyether having a branching structure that repeats regularly with each successive generation of monomer, radiating from a core molecule.

The term “comb polyether” refers to a multi-arm polyether in which linear side chains emanate from trifunctional branch points on a linear polymer backbone.

The term “star polyether” refers to a multi-arm polyether in which linear side chains emanate from a single atom or a core molecule having a point of symmetry.

The term “highly branched polyether” refers to a multi-arm polyether having many branch points, such that the distance between branch points is small relative to the total length of the arms.

The term “hyperbranched polyether” refers to a multi-arm polyether that is more branched than highly branched with order approaching that of an imperfect dendritic polyether.

The term “branched end amine” refers to a linear or multi-arm polymer having two or three primary amine groups at each of the ends of the polymer chain or at the end of the polymer arms.

The term “multi-functional amine” refers to a chemical compound having two or more functional groups, at least one of which is a primary amine.

The term “% by weight” as used herein refers to the weight percent relative to the total weight of the solution or dispersion, unless otherwise specified.

The term “anatomical site” refers to any external or internal part of the body of humans or animals.

The term “tissue” refers to any tissue, both living and dead, in humans or animals.

The term “hydrogel” refers to a water-swellable polymeric matrix, consisting of a three-dimensional network of macromolecules held together by covalent or non-covalent crosslinks, that can absorb a substantial amount of water to form an elastic gel.

The term “gelation time”, also referred to herein as “the gelation time of a/the hydrogel” refers to the time for the combination of two or more components to react to form a hydrogel, which when stirred, no longer flows and hold its shape.

The term “degradation time” refers to the time at which a hydrogel dissolves, as determined by visual inspection, when incubated in an aqueous medium with shaking at a specified temperature and agitation speed. For example, the degradation time may be determined by incubating the hydrogel in phosphate-buffered saline (PBS) with agitation at 85 rpm at a temperature of 37° C.

The term “time-to-tack-free” refers to the time at which a hydrogel adhesive no longer forms a visually discernible bond immediately on contact with a solid object, such as a spatula, or with itself.

The term “biocompatible”, as used herein, refers to a chemical additive that is nontoxic to biological tissue when used at concentrations needed to extend the gelation time of a hydrogel formed by reacting an oxidized polysaccharide and a multi-arm amine according to the method disclosed herein.

The term “crosslink” refers to a bond or chain attached between and linking two different polymer chains.

The term “crosslink density” is herein defined as the reciprocal of the average number of chain atoms between crosslink connection sites.

By medical application is meant medical applications as related to humans or animals.

### Oxidized Polysaccharides:

Polysaccharides useful in the invention include, but are not limited to, dextran, starch, agar, cellulose, hydroxyethylcellulose, pullulan, and hyaluronic acid. These polysaccharides are available commercially from sources such as Sigma Chemical Co. (St. Louis, Mo.). In one embodiment, the polysaccharide is dextran. Typically, polysaccharides are a heterogeneous mixture having a distribution of different molecular weights, and are characterized by an average molecular weight, for example, the weight-average molecular weight (M_{w}), or the number average molecular weight (Mₙ), as is known in the art. Suitable polysaccharides have a weight-average molecular weight from about 1,000 to about 1,000,000 Daltons, and in addition from about 3,000 to about 250,000 Daltons.

The polysaccharide is oxidized to introduce aldehyde groups using any suitable oxidizing agent, including but not limited to, periodates, hypochlorites, ozone, peroxides, hydroperoxides, persulfates, and percarbonates. In one embodiment, the polysaccharide is oxidized by reaction with sodium periodate, for example as described by Mo et al. (J. Biomater. Sci. Polymer Edn. 11:341-351, 2000). The polysaccharide is reacted with different amounts of periodate to give polysaccharides with different degrees of oxidation and therefore, different amounts of aldehyde groups, as described in detail in the General Methods Section of the Examples infra. The aldehyde content of the oxidized polysaccharide may be determined using methods known in the art. For example, the dialdehyde content of the oxidized polysaccharide may be determined using the method described by Hofreiter et al. (Anal Chem. 27:1930-1931, 1955), as described in detail in the General Methods Section of the Examples infra. In that method, the amount of alkali consumed per mole of dialdehyde in the oxidized polysaccharide, under specific reaction conditions, is determined by a pH titration. In one embodiment, the equivalent weight per aldehyde group of the oxidized polysaccharide is from about 90 to about 1500 Daltons.

In the methods disclosed herein, the oxidized polysaccharide is typically used in the form of an aqueous solution or dispersion. However, the oxidized polysaccharide need not be used in the form of aqueous solution or dispersion. The presence of water is optional. For example, the oxidized polysaccharide may be used in dry form in the presence of water or an aqueous body fluid, as described by Sawhney et al. (U.S. Pat. No. 6,703,047) and Odermatt et al. (U.S. Patent Application Publication No. 2006/0134185, both of which are incorporated herein by reference.

In one embodiment, at least one oxidized polysaccharide is used in the form of an aqueous solution or dispersion. The oxidized polysaccharide is added to water to give a concentration of about 5% to about 40%, in addition from about 5% to about 30%, and in addition from about 15% to about 30% by weight relative to the total weight of the solution. The optimal concentration to be used depends on the application and on the concentration of the multi-arm amine used, as described infra, and can be readily determined by one skilled in the art using routine experimentation. Additionally, a mixture of at least two different oxidized polysaccharide distributions having different weight-average molecular weights, different degrees of oxidation, or different weight-average molecular weights and different degrees of oxidation may be used. Where a mixture of oxidized polysaccharide distributions is used, the total concentration of the oxidized polysaccharides is about 5% to about 40% by weight, in addition from about 15% to about 30% by weight relative to the total weight of the solution.

For use on living tissue, it is preferred that the aqueous solution or dispersion comprising the oxidized polysaccharide be sterilized to prevent infection. Any suitable sterilization method known in the art that does not degrade the polysaccharide may be used, including, but not limited to, electron beam irradiation, gamma irradiation, ethylene oxide sterilization, or ultra-filtration through a 0.2 μm pore membrane.

The aqueous solution or dispersion comprising the oxidized polysaccharide may further comprise various adjuvants depending on the intended application. Preferably, the adjuvant is compatible with the oxidized polysaccharide. Specifically, the adjuvant does not contain primary or secondary amine groups that would interfere with effective gelation to form a hydrogel. The amount of the adjuvant used depends on the particular application and may be readily determined by one skilled in the art using routine experimentation. For example, the aqueous solution or dispersion comprising the oxidized polysaccharide may optionally include at least one thickener. The thickener may be selected from among known viscosity modifiers, including, but not limited to, polysaccharides and derivatives thereof, such as starch or hydroxyethyl cellulose.

The aqueous solution or dispersion comprising the oxidized polysaccharide may optionally include at least one antimicrobial agent. Suitable antimicrobial preservatives are well known in the art. Examples of suitable antimicrobials include, but are not limited to, alkyl parabens, such as methylparaben, ethylparaben, propylparaben, and butylparaben; triclosan; chlorhexidine; cresol; chlorocresol; hydroquinone; sodium benzoate; and potassium benzoate.

The aqueous solution or dispersion comprising the oxidized polysaccharide may also optionally include at least one colorant to enhance the visibility of the solution or dispersion. Suitable colorants include dyes, pigments, and natural coloring agents. Examples of suitable colorants include, but are not limited to, FD&C Violet No. 2, FD&C Blue No. 1, D&C Green No. 6, D&C Green No. 5, D&C Violet No. 2; and natural colorants such as beetroot red, canthaxanthin, chlorophyll, eosin, saffron, and carmine.

The aqueous solution or dispersion comprising the oxidized polysaccharide may also optionally include at least one surfactant. Surfactant, as used herein, refers to a compound that lowers the surface tension of water. The surfactant may be an ionic surfactant, such as sodium lauryl sulfate, or a neutral surfactant, such as polyoxyethylene ethers, polyoxyethylene esters, and polyoxyethylene sorbitan.

Additionally, the aqueous solution or dispersion comprising the oxidized polysaccharide may optionally include a pharmaceutical drug or therapeutic agent, including but not limited to, antibacterial agents, antiviral agents, antifungal agents, anti-cancer agents, vaccines, radiolabels, anti-inflammatory agents, such as indomethacin, salicylic acid acetate, ibuprofen, sulindac, piroxicam, and naproxen; thrombogenic agents, such as thrombin, fibrinogen, homocysteine, and estramustine; and radio-opaque compounds, such as barium sulfate and gold particles.

### Water-Dispersible, Multi-Arm Amines:

Suitable water dispersible, multi-arm amines include, but are not limited to, water dispersible multi-arm polyether amines, amino-terminated dendritic polyamidoamines, and multi-arm branched end amines.

Typically, the multi-arm amines have a number-average molecular weight of about 450 to about 200,000 Daltons, in addition from about 2,000 to about 40,000 Daltons.

In one embodiment, the water dispersible, multi-arm amine is a multi-arm polyether amine, which is a water-dispersible polyether having the repeat unit [—O—R]—, wherein R is a hydrocarbylene group having 2 to 5 carbon atoms. The term “hydrocarbylene group” refers to a divalent group formed by removing two hydrogen atoms, one from each of two different carbon atoms, from a hydrocarbon. Suitable multi-arm polyether amines include, but are not limited to, dendritic, comb, star, highly branched, and hyperbranched polyethers wherein at least three of the arms are terminated by a primary amine group. Examples of water-dispersible, multi-arm polyether amines include, but are not limited to, amino-terminated star, dendritic, or comb polyethylene oxides; amino-terminated star, dendritic or comb polypropylene oxides; amino-terminated star, dendritic or comb polyethylene oxide-polypropylene oxide copolymers; and polyoxyalkylene triamines, sold under the trade name Jeffamine® triamines, by Huntsman LLC. (Houston, Tex.). Examples of star polyethylene oxide amines, include, but are not limited to, various multi-arm polyethylene glycol amines, available from Nektar Transforming Therapeutics (Huntsville, Ala.), and star polyethylene glycols having 3, 4, 6, or 8 arms terminated with primary amines (referred to herein as 3, 4, 6, or 8-arm star PEG amines, respectively). The 8-arm star PEG amine is available from Nektar Transforming Therapeutics. Examples of suitable Jeffamine® triamines include, but are not limited to, Jeffamine® T-403 (CAS No. 39423-51-3), Jeffamine® T-3000 (CAS No. 64852-22-8), and Jeffamine® T-5000 (CAS No. 64852-22-8). In one embodiment, the water-dispersible multi-arm polyether amine is an eight-arm polyethylene glycol having eight arms terminated by a primary amine group and having a number-average molecular weight of about 10,000 Daltons (available from Nektar Transforming Therapeutics).

The multi-arm polyether amines are either available commercially, as noted above, or may be prepared using methods known in the art. For example, multi-arm polyethylene glycols, wherein at least three of the arms are terminated by a primary amine group, may be prepared by putting amine ends on multi-arm polyethylene glycols (e.g., 3, 4, 6, and 8-arm star polyethylene glycols, available from companies such as Nektar Transforming Therapeutics; SunBio, Inc., Anyang City, South Korea; NOF Corp., Tokyo, Japan; or JenKem Technology USA, Allen, Tex.) using the method described by Buckmann et al. (Makromol. Chem. 182:1379-1384, 1981). In that method, the multi-arm polyethylene glycol is reacted with thionyl bromide to convert the hydroxyl groups to bromines, which are then converted to amines by reaction with ammonia at 100° C. The method is broadly applicable to the preparation of other multi-arm polyether amines. Additionally, multi-arm polyether amines may be prepared from multi-arm polyols using the method described by Chenault (copending and commonly owned U.S. Patent Application Publication No. 2007/0249870). In that method, the multi-arm polyether is reacted with thionyl chloride to convert the hydroxyl groups to chlorine groups, which are then converted to amines by reaction with aqueous or anhydrous ammonia. Other methods that may used for preparing multi-arm polyether amines are described by Merrill et al. in U.S. Pat. No. 5,830,986, and by Chang et al. in WO 97/30103.

The multi-arm amine may also be an amino-terminated dendritic polyamidoamine, sold under the trade name Starburst® Dendrimers (available from Sigma-Aldrich, St Louis, Mo.).

The multi-arm amine may also be a multi-arm branched end amine, as described by Arthur (copending and commonly owned Patent Application No. PCT/U.S.07/24393). The branched end amines can be linear or branched polymers having two or three amine groups at each of the ends of the polymer chain or at the end of the polymer arms. The multiplicity of functional groups increases the statistical probability of reaction at a given chain end and allows more efficient incorporation of the linear or branched molecules into a polymer network. The starting materials used to prepare the branched end amines may be linear polymers such as polyethylene oxide, poly(trimethyleneoxide), block or random copolymers of polyethylene oxide and polypropylene oxide or triblock copolymers of polyethylene oxide and polypropylene oxide, having terminal hydroxyl groups, or branched polymers such as multi-arm polyether polyols including, but not limited to, comb and star polyether polyols. The branched end amines can be prepared by attaching multiple amine groups to the ends of the polymer by reaction with the hydroxyl groups using methods well known in the art. For example, a branched end amine having two amine functional groups on each end of the polymer chain or at the end of the polymer arms can prepared by reacting the starting material, as listed above, with thionyl chloride in a suitable solvent such as toluene to give the chloride derivative, which is subsequently reacted with tris(2-aminoethyl)amine to give the branched end reactant having two amino groups at each end of the polymer chain or arm. In one embodiment, the branched end amine is an 8-arm polyethylene glycol (PEG) hexadecaamine, having a weight-average molecular weight of about 40,000 Daltons, prepared as described in the General Methods section of the Examples, infra.

It should be recognized that the multi-arm amines are generally a somewhat heterogeneous mixture having a distribution of arm lengths and in some cases, a distribution of species with different numbers of arms. When a multi-arm amine has a distribution of species having different numbers of arms, it can be referred to based on the average number of arms in the distribution. For example, in one embodiment the multi-arm amine is an 8-arm star PEG amine, which comprises a mixture of multi-arm star PEG amines, some having less than and some having more than 8 arms; however, the multi-arm star PEG amines in the mixture have an average of 8 arms. Therefore, the terms “8-arm”, “6-arm”, “4-arm” and “3-arm” as used herein to refer to multi-arm amines, should be construed as referring to a heterogeneous mixture having a distribution of arm lengths and in some cases, a distribution of species with different numbers of arms, in which case the number of arms recited refers to the average number of arms in the mixture.

In the methods disclosed herein, the multi-arm amine is typically used in the form of an aqueous solution or dispersion. However, the multi-arm amine need not be used in the form of an aqueous solution or dispersion. The presence of water is optional. For example, some multi-arm amines are liquids, which may be used neat. Additionally, the multi-arm amine may be used in dry form in the presence of water or an aqueous body fluid, as described by Sawhney et al. (U.S. Pat. No. 6,703,047) and Odermatt et al. (U.S. Patent Application Publication No. 2006/0134185, both of which are incorporated herein by reference.

In one embodiment, at least one multi-arm amine is used in the form of an aqueous solution or dispersion. The multi-arm amine is added to water to give a concentration of about 5% to about 70% by weight, in addition from about 20% to about 50% by weight relative to the total weight of the solution. The optimal concentration to be used depends on the application and on the concentration of the oxidized polysaccharide used. Additionally, a mixture of different multi-arm amine distributions having different number-average molecular weights, different numbers of arms, or different number-average molecular weights and different numbers of arms may be used. Where a mixture of multi-arm amine distributions is used, the total concentration of the multi-arm amines is about 5% to about 70% by weight, in addition from about 20% to about 50% by weight relative to the total weight of the solution.

In one embodiment, the concentrations of the oxidized polysaccharide and the multi-arm amine are adjusted such that the aldehyde groups on the oxidized polysaccharide are in stoichiometric excess relative to the amine groups on the multi-arm amine. In one embodiment, wherein an 8-arm star PEG amine is used as the multi-arm amine, the amount of aldehyde groups is from about 1.1 times to about 50 times the amount of amine groups, in addition from about 3 times to about 15 times the amount of amine groups. In another embodiment wherein a Jeffamine® triamine is used as the multi-arm amine, the amount of aldehyde groups is from about 0.5 times to about 3 times the amount of amine groups.

For use on living tissue, it is preferred that the aqueous solution or dispersion comprising the multi-arm amine be sterilized to prevent infection. Any of the methods described above for sterilizing the oxidized polysaccharide solution may be used.

The aqueous solution or dispersion comprising the multi-arm amine may further comprise various adjuvants. Any of the adjuvants described above for the oxidized polysaccharide solution may be used. Additionally, the solution may comprise a healing promoter, such as chitosan.

Additionally, the aqueous solution or dispersion comprising the multi-arm amine may optionally comprise at least one other multi-functional amine having one or more primary amine groups to provide other beneficial properties, such as hydrophobicity or modified crosslink density. The multi-functional amine is capable of inducing gelation when mixed with an oxidized polysaccharide in an aqueous solution or dispersion. The multi-functional amine may be a second water dispersible, multi-arm amine, such as those described above, or another type of multi-functional amine, including, but not limited to, linear and branched diamines, such as diaminoalkanes, polyaminoalkanes, and spermine; linear branched end amines as described above, branched polyamines, such as polyethylenimine; cyclic diamines, such as N,N′-bis(3-aminopropyl)piperazine, 5-amino-1,3,3-trimethylcyclohexanemethylamine, 1,3-bis(aminomethyl)cyclohexane, 1,4-diaminocyclohexane, and p-xylylenediamine; aminoalkyltrialkoxysilanes, such as 3-aminopropyltrimethoxysilane and 3-aminopropyltriethoxysilane; aminoalkyldialkoxyalkylsilanes, such as 3-aminopropyldiethoxymethylsilane, dihydrazides, such as adipic dihydrazide; linear polymeric diamines, such as linear polyethylenimine, α,ω-amino-terminated polyethers, α,ω-bis(3-aminopropyl)polybutanediol, β,ω-1-amino-terminated polyethers (linear Jeffamines®); comb polyamines, such as chitosan, polyallylamine, and polylysine, and di- and polyhydrazides, such as bis(carboxyhydrazido)polyethers and poly(carboxyhydrazido) star polyethers. Many of these compounds are commercially available from companies such as Sigma-Aldrich and Huntsman LLC. Typically, if present, the multi-functional amine is used at a concentration of about 5% by weight to about 1000% by weight relative to the weight of the multi-arm amine in the aqueous solution or dispersion.

In another embodiment, the multi-functional amine is provided in a separate solution at a concentration of about 5% by weight to about 100% by weight relative to the total weight of the solution. If the multi-functional amine is not used neat (i.e., 100% by weight), it is used in the form of an aqueous solution or dispersion. For use on living tissue, it is preferred that the solution comprising the multi-functional amine be sterilized. Any of the methods described above for sterilizing the oxidized polysaccharide solution may be used. The aqueous solution or dispersion comprising the multi-functional amine may further comprise various adjuvants. Any of the adjuvants described above for the oxidized polysaccharide solution or the multi-arm amine solution may be used.

### Chemical Additives

The chemical additive is biocompatible, has a molecular weight of less than about 2,000 Daltons, and comprises at least one reactive group capable of reacting with an amine or an aldehyde group. However, the additive is not capable of inducing gelation, under the conditions of use, when mixed in an aqueous medium with either an oxidized polysaccharide alone or a multi-arm amine alone, even though the chemical additive may comprise more than one reactive group capable of reacting with aldehyde or amine groups. For example, for a chemical additive comprising more than one reactive group which is capable of reacting with amine or aldehyde groups, the reaction of all but one of the reactive groups may be sterically hindered. Additionally, the chemical additive comprising more than one reactive group may be used at a low concentration so that gelation is not induced; or only one of the reactive groups may be reactive at the conditions used.

Reactive groups that are capable of reacting with amine or aldehyde groups are well known in the art. For example, reactive groups that are capable of reacting with amine groups include, but are not limited to, electrophilic groups such as aldehyde, ketone, glyoxal, acetoacetate, activated ester, imidoester, maleimide, p-nitrophenyl ester, activated halide, anhydride, carbonyl imidazole, epoxide, alkylhalide, and H⁺. Reactive groups that are capable of reacting with aldehyde groups include, but are not limited to, nucleophilic groups such as primary amine, secondary amine, and carboxyhydrazide groups, and OH⁻.

Suitable chemical additives include, but are not limited to, primary amines, such as glucosamine and 2-aminoethanol; secondary amines, such as diisopropylamine; aldose sugars, such as D-glucose and D-mannose; ketose sugars, such as D-ribulose, D-fructose, D-glyceraldehyde, and dihydroxyacetone; Brønsted acids, such as hydrochloric acid, acetic acid, and carboxylic acids; acid salts, such as glucosamine hydrochloride and 2-aminoethanol hydrochloride; Brønsted bases such as sodium hydroxide and potassium hydroxide; amino acids, such as lysine, cysteine, arginine, and serine; short peptides having 2 to about 15 amino acids, such as the peptide given as SEQ ID NO:1; activated esters, such as N-hydroxysuccinimidyl ester, sulfo-succinimidyl acetate, and methyl acetimidate hydrochloride; and activated halides, such as allyl chloride, benzyl bromide, butyryl chloride, and 2,4-dinitrofluorobenzene. In a preferred embodiment, the chemical additive is at least one additive selected from the group consisting of glucosamine, 2-aminoethanol, diisopropylamine, D-glucose, hydrochloric acid, acetic acid, glucosamine hydrochloride, 2-aminoethanol hydrochloride, sodium hydroxide, lysine, cysteine, serine, and a peptide having a sequence as set forth in SEQ ID NO:1.

The chemical additive reacts with either the aldehyde groups of the oxidized polysaccharide or the amine groups of the multi-arm amine, thereby reducing the number of functional groups available for crosslinking to form the hydrogel. The bond formed in the reaction of the chemical additive with the aldehyde or amine groups may be reversible or irreversible. If the bond is reversible, the rate of crosslinking is decreased, resulting in an extended gelation time and time-to-tack-free, but the overall crosslink density may not be affected because the chemical additive may be displaced by one of the crosslinkable components (i.e., either the oxidized polysaccharide or the multi-arm amine). If the bond is irreversible, the rate of crosslinking is decreased resulting in an extended gelation time and time-to-tack-free and the crosslink density is decreased resulting in a decreased degradation time for the resulting hydrogel. The adhesive and/or cohesive strength of the hydrogel may also be decreased by the chemical additive, so that the gelation time, time-to-tack-free, degradation time, and adhesive/cohesive strength will need to be optimized for any given application. This optimization may be done by one skilled in the art using routine experimentation.

### Method for Extending Gelation Time:

In one embodiment, the invention provides a method for extending the gelation time for at least one oxidized polysaccharide (component A), as described above, and at least one water-dispersible, multi-arm amine (component B), as described above, to form a hydrogel in an aqueous medium under predetermined conditions. The method comprises contacting component A and component B in the presence of an aqueous medium and at least one chemical additive, as described above, to form a mixture that forms a resulting hydrogel. The chemical additive is used in an amount sufficient to extend the gelation time of components (A) and (B) under predetermined conditions by at least about 10% compared to that of components (A) and (B) under the same conditions, but in the absence of the additive. The time-to-tack-free is also extended. The contacting of components A and B and the chemical additive may be on an anatomical site on tissue of a living organism to form the mixture and the resulting hydrogel directly on the site. Additionally, the mixture, after it is formed by contacting the components, may be applied to the anatomical site to form the resulting hydrogel on the site.

As described above, the oxidized polysaccharide, the multi-arm amine, and the chemical additive are typically provided in the form of aqueous solutions or dispersions which provide the aqueous medium for the formation of the hydrogel. However, the components may also be provided in dry form and the aqueous medium may be a body fluid, as described above. If the components are provided in dry form, the chemical additive may be added to either the oxidized polysaccharide or the multi-arm amine component in an aqueous solution or dispersion to facilitate reaction between the chemical additive and the desired component, and then the resulting mixture is dried. Alternatively, an aqueous solution comprising the chemical additive may be applied to at least one of the dried components, for example, by spraying. Additionally, the hydrogel components may be provided in dry forms that are reconstituted with water prior to use,

The oxidized polysaccharide and the multi-arm amine, when mixed under predetermined conditions in an aqueous medium react to form a hydrogel. The predetermined conditions include: the concentrations of the oxidized polysaccharide and the multi-arm amine, the weight-average molecular weight of the oxidized polysaccharide, the number-average molecular weight of the multi-arm amine, the degree of oxidation of the polysaccharide, the ratio of aldehyde to amine groups, the temperature of the reaction, the agitation rate, and the like. For any set of predetermined conditions, the gelation time and the time-to-tack-free can be determined using methods known in the art. The addition of a sufficient amount of the chemical additive under the same predetermined conditions results in extending the gelation time by at least about 10%.

The gelation time can be measured by a variety of different methods. For example, the aqueous solution or dispersion comprising the oxidized polysaccharide and the aqueous solution or dispersion comprising the multi-arm amine can be combined with stirring and the time it takes for the mixture to gel to the point where it holds its shape without flowing can be measured. A more precise measurement of gelation time may be performed by oscillating disk rheometry. The gel point is the time at which the values of G′ (the elastic or storage modulus) and G″ (the viscous or loss modulus) are equal. Similarly, the time-to-tack-free can also be measured in a variety of ways. For example, the aqueous solution or dispersion comprising the oxidized polysaccharide and the aqueous solution or dispersion comprising the multi-arm amine can be combined with stirring, and the time it takes for the mixture to gel to the point where the resulting hydrogel does not bond to a solid object, such as a spatula, can be measured.

The chemical additive may be added to at least one of the following solutions or dispersions: the aqueous solution or dispersion comprising the oxidized polysaccharide, the aqueous solution or dispersion comprising the multi-arm-polyether amine, or a third aqueous solution. The third aqueous solution may be sterilized using any of the methods described above and may further comprise various adjuvants, as described above for the aqueous solution comprising the oxidized polysaccharide. In one embodiment, the chemical additive is added to the aqueous solution or dispersion comprising the crosslinkable component that comprises functional groups that react with the at least one reactive group of the chemical additive. For example, if the chemical additive comprises at least one reactive group that is capable of reacting with amine groups, the additive is added to the aqueous solution or dispersion comprising the multi-arm amine. Conversely, if the chemical additive comprises at least one functional group that is capable of reacting with aldehyde groups, the additive is added to the aqueous solution or dispersion comprising the oxidized polysaccharide. The chemical additive is chosen so that it does not induce gelation when added to the aqueous solution or dispersion comprising the multi-arm amine or the aqueous solution or dispersion comprising the oxidized polysaccharide. In some embodiments where the reaction of the chemical additive with the crosslinkable component is slow, it may be preferable to preincubate the mixture to allow sufficient time for the reaction to take place before adding the second crosslinkable component to form the hydrogel.

The chemical additive is provided in a sufficient amount to provide the desired extension of gelation time. In general, the larger amount of the chemical additive used, the greater is the effect on extending the gelation time and the time-to-tack-free of the resulting hydrogel. The amount of the chemical additive to be used to achieve the desired properties can be determined by one skilled in the art using routine experimentation. Typically, the chemical additive is used in an amount such that the mole ratio of the at least one reactive group of the chemical additive relative to the amine or aldehyde groups with which the reactive group reacts is between about 0.1 and about 1.5, in addition between about 0.15 and about 1.0, and in addition between about 0.20 and about 0.80.

In one embodiment, the chemical additive is provided in at least one of the aqueous solution or dispersion comprising the oxidized polysaccharide, referred to herein as the “first aqueous solution or dispersion”, or the aqueous solution or dispersion comprising the multi-arm amine, referred to herein as the “second aqueous solution or dispersion”. The solutions or dispersions may be applied to an anatomical site on tissue of a living organism in any number of ways. Once both solutions are combined on a site, they crosslink to form a hydrogel. Because the aldehyde groups of the oxidized polysaccharide may also covalently bind to amine groups on the tissue, the hydrogel adhesive of the invention is capable of covalently binding to tissue, thereby contributing to its adhesive strength.

In one embodiment, the two aqueous solution or dispersions are applied to the site sequentially using any suitable means including, but not limited to, spraying, brushing with a cotton swab or brush, or extrusion using a pipet, or a syringe. The solutions may be applied in any order. Then, the solutions are mixed on the site using any suitable device, such as a cotton swab, a spatula, or the tip of the pipet or syringe.

In another embodiment, the two aqueous solution or dispersions are mixed manually in a suitable vessel (e.g., a tube, vial, or the like) before application to the site. The resulting mixture is then applied to the site before it completely cures using a suitable applicator, as described above.

In another embodiment, the two aqueous solution or dispersions are contained in a double-barrel syringe. In this way the two aqueous solution or dispersions are applied simultaneously to the site with the syringe. Suitable double-barrel syringe applicators are known in the art. For example, Redl describes several suitable applicators for use in the invention in U.S. Pat. No. 6,620,125, (particularly FIGS. 1, 5, and 6, which are described in Columns 4, line 10 through column 6, line 47) which is incorporated herein by reference. Additionally, the double barrel syringe may contain a motionless mixer, such as that available from ConProtec, Inc. (Salem, N.H.) or MixPac (Rotkreutz, Switzerland), at the tip to effect mixing of the two aqueous solution or dispersions prior to application.

In another embodiment, the two aqueous solutions or dispersions may be applied to the site using a spray device, such as those described by Fukunaga et al. (U.S. Pat. No. 5,582,596) or Sawhney (U.S. Pat. No. 6,179,862).

In another embodiment, the two aqueous solutions or dispersions may be applied to the site using a minimally invasive surgical applicator, such as those described by Sawhney (U.S. Pat. No. 7,347,850).

In another embodiment, the chemical additive is provided in a third aqueous solution. The three solutions are applied to the anatomical site in any order using any of the methods described above. In this embodiment, the double-barrel syringe may be modified to have three barrels, one for each of the solutions.

### Method for Decreasing Degradation Time:

In another embodiment, the invention provides a method for decreasing the degradation time of a hydrogel formed from at least one oxidized polysaccharide (component A), as described above, and at least one water-dispersible multi-arm amine (component B), as described above, in an aqueous medium to form a hydrogel under predetermined conditions. The method comprises contacting component A and component B in the presence of an aqueous medium and at least one chemical additive, as described above, to form a mixture that forms a resulting hydrogel. The at least one chemical additive is used in an amount sufficient to decrease the gelation time of the resulting hydrogel under the predetermined conditions by at least about 10% compared to that of the hydrogel formed under said conditions, but in the absence of the additive.

To provide a decreased degradation time, a chemical additive that forms an irreversible bond with either the aldehyde groups of the oxidized polysaccharide or the amine groups of the multi-arm amine is typically used, although chemical additives that form reversible bonds may also provide a decreased degradation time. The at least one oxidized polysaccharide, the at least one water-dispersible, multi-arm amine, and the at least one chemical additive are contacted in an aqueous medium to form a mixture that forms a resulting hydrogel having a degradation time that is decreased by at least about 10% compared to that of the hydrogel formed in the absence of the chemical additive, as described above. The hydrogel components are typically provided in the form of aqueous solutions or dispersions which provide the aqueous medium for the formation of the hydrogel. However, one or more of the components may also be provided in dry form, as described above.

For any set of predetermined conditions, the degradation time of the resulting hydrogel can be determined using methods known in the art. The addition of the chemical additive under the same predetermined conditions results in decreasing the degradation time by at least about 10%. The degradation time of the hydrogel can be measured using methods known in the art. For example, after the hydrogel is formed by mixing the aqueous solution or dispersion comprising the oxidized polysaccharide and the aqueous solution or dispersion comprising the multi-arm amine, it can be incubated in an aqueous medium with shaking at a specified temperature and agitation speed and the time required for the gel to dissolve can be measured. Typical conditions for measuring the degradation rate of a hydrogel may be incubation in PBS buffer with agitation at 85 rpm at a temperature of 37° C.

The at least one oxidized polysaccharide, the at least water-dispersible multi-arm amine, and the at least one chemical additive are contacted in the presence of an aqueous medium and applied to an anatomical site on tissue of a living organism as described above for the method for extending gelation time.

### Medical and Veterinary Applications:

Hydrogels prepared by the method of the invention have many potential medical and veterinary applications, such as those described by Kodokian et al., supra, which is incorporated herein by reference. Examples include, but are not limited to, wound closure, supplementing or replacing sutures or staples in internal surgical procedures such as intestinal anastomosis and vascular anastomosis, ophthalmic procedures, drug delivery, and anti-adhesive applications.

The method of the invention is particularly useful for preparing hydrogels for applications in which an extended gelation time or a decreased degradation time is desirable. For example, an extended gelation time is beneficial in an intestinal anastomosis procedure wherein sufficient time is needed for the application of the mixed hydrogel components around the entire circumference of the intestine to form a complete seal. If the mixed components gel too quickly, the entire anastomosis site may not be sealed properly due to poor application, clogging of the applicator, or failure of the hydrogel to bond to itself once it cures. Additionally, slower gelation is desirable for use in minimally invasive surgeries, such as laparoscopic surgery, where the mixed hydrogel components are delivered by means of a long tube. Sufficient time is required for the mixed components to reach to the site before gelation occurs. An example of an application where a decreased degradation time may be desirable is for use of the hydrogel for adhesion prevention, wherein the hydrogel should not persist at the site once the healing process has begun.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

The meaning of abbreviations used is as follows: the designation “10K” means that a polymer molecule possesses an average molecular weight of 10 kiloDaltons, a designation of “60K” indicates an average molecular weight of 60 kiloDaltons, etc, “min” means minute(s), “h” means hour(s), “sec” means second(s), “d” means day(s), “mL” means milliliter(s), “L” means liter(s), “μL” means microliter(s), “cm” means centimeter(s), “mm” means millimeter(s), “μm” means micrometer(s), “mol” means mole(s), “mmol” means millimole(s), “g” means gram(s), “mg” means milligram(s), “meq” means milliequivalent(s), “EW” means equivalent weight, “M_{w}” means weight-average molecular weight, “Mₙ” means number-average molecular weight, “M_{z}” means z-average molecular weight, “MW” means molecular weight, “wt %” means percent by weight, “mol %” means mole percent, “Vol” means volume, “v/v” means volume per volume, “EO” means ethylene oxide, “PO” means propylene oxide, “PEG” means polyethylene glycol, “Da” means Daltons, “kDa” means kiloDaltons, “MWCO” means molecular weight cut-off, “¹H NMR” means proton nuclear magnetic resonance spectroscopy, “ppm” means parts per million, “D” means density in g/mL, “Vol” means volume, “rpm” means revolutions per minute, “PBS” means phosphate-buffered saline, “dn/dc” means the specific refractive index increment (i.e., the change in refractive index per change in concentration), “IV” means intrinsic viscosity, “MHz” means megahertz, “SEC” means size exclusion chromatography, “DMSO-d₆” means deuterated dimethyl sulfoxide, and “Ac” means an acetate group.

A reference to “Aldrich” or a reference to “Sigma” means the said chemical or ingredient was obtained from Sigma-Aldrich, St. Louis, Mo. A reference to “Shearwater” or “Nektar” means the said chemical or ingredient was obtained from Nektar, Huntsville, Ala. A reference to “SunBio” means the said chemical or ingredient was obtained from SunBio Inc., Anyang City, South Korea. A reference to “NOF” means the said chemical or ingredient was obtained from NOF Corp, Tokyo, Japan. A reference to “TCI America” means the said chemical or ingredient was obtained from TCI America, Portland, Oreg. A reference to “BASF” means the said chemical or ingredient was obtained from BASF Corp, Ludwigshafen, Germany.

### General Methods:

### Preparation of Oxidized Dextran

The following procedure was used to prepare an oxidized dextran, also referred to herein as dextran aldehyde, with about 50% aldehyde content conversion from dextran having an average molecular weight of 8,500-11,500 Daltons. Other aldehyde conversions were obtained by varying the concentration of the periodate solution used. Likewise dextrans of other molecular weights (i.e., average molecular weight of 60,000 to 90,000, Sigma # D3759) were oxidized to provide the corresponding oxidized dextran.

Dextran (19.0 g; 0.12 mol saccharide rings; average molecular weight 8,500-11,500; Sigma # D9260) was added to 170 g of distilled water in a 500 mL round bottom flask. The mixture was stirred for 15 to 30 min to produce a solution; then a solution of 17.7 g (0.083 mol; MW=213.9) sodium periodate in 160 g of distilled water was added to the dextran solution all at once. The mixture was stirred at room temperature for 5 h. After this time, the solution was removed from the round bottom flask, divided into four equal volumes and dispensed into 4 dialysis membrane tubes (MWCO=3500 Daltons). The tubes were dialyzed in deionized water for 4 days, during which time the water was changed twice daily. The aqueous solutions were removed from the dialysis tubes, placed in wide-mouth polyethylene containers and frozen using liquid nitrogen, and lyophilized to afford white, fluffy oxidized dextran.

The dialdehyde content in the resulting oxidized dextran was determined using the following procedure. The oxidized dextran (0.1250 g) was added to 10 mL of 0.25 M NaOH in a 250 mL Erlenmeyer flask. The mixture was gently swirled and then placed in a temperature-controlled sonicator bath at 40° C. for 5 min until all the material dissolved, giving a dark yellow solution. The sample was removed from the bath and the flask was cooled under cold tap water for 5 min. Then 15.00 mL of 0.25 M HCl was added to the solution, followed by the addition of 50 mL of distilled water and 1 mL of 0.2% phenolphthalein solution. This solution was titrated with 0.25 M NaOH to an endpoint determined by a color change from yellow to purple/violet. The same titration was carried out on a sample of the starting dextran to afford a background aldehyde content. The dialdehyde content, also referred to herein as the oxidation conversion or the degree of oxidation, in the oxidized dextran sample was calculated using the following formula:

$$Dialdehyde Content = \frac{{( Vb - Va )}_{s}}{{W}_{s} / M} - \frac{{( Vb - Va )}_{p}}{{W}_{p} / M} \times 100 \%$$

Vb=total meq of base
Va=total meq of acid
W=dry sample weight (mg)
M=weight-average molecular weight of polysaccharide repeat unit (=162 for dextran)
s=oxidized sample
p=original sample

Typically, three determinations were done and the degree of oxidation given in the following Examples is the mean of the three determinations.

### Preparation of 8-Arm Polyethylene Glycol 10K Octaamine

An 8-arm PEG octaaamine was synthesized using the two-step procedure described by Chenault in co-pending and commonly owned U.S. Patent Application Publication No. 2007/0249870.

Eight-arm star PEG-OH, Mₙ 10,000 (determined by hydroxyl end group titration assuming all the polymer molecules have eight arms), was obtained from NOF America Corp. (White Plains, N.Y.). The 8-arm star PEG-OH (100 g in a 500-mL round-bottom flask) was dried either by heating with stirring at 85° C. under vacuum (0.06 mm of mercury (8.0 Pa)) for 4 h or by azeotropic distillation with 50 g of toluene under reduced pressure (15 mm of mercury (2 kPa)) with a pot temperature of 60° C.

The 8-arm star PEG-OH was allowed to cool to room temperature. Then, thionyl chloride (35 mL, 0.48 mol) was added to the flask, which was equipped with a reflux condenser, and the mixture was heated at 85° C. with stirring under a blanket of nitrogen for 24 h. Excess thionyl chloride was removed by rotary evaporation (bath temp 40° C.). Two successive 50-mL portions of toluene were added and evaporated under reduced pressure (15 mm of mercury (2 kPa), bath temperature 60° C.) to complete the removal of thionyl chloride. The yield of 8-arm star PEG-Cl was 100.9 g (99%).

¹H NMR (500 MHz, DMSO-d₆) δ 3.71-3.69 (m, 16H), 3.67-3.65 (m, 16H), 3.50 (s, ˜800H). Aqueous SEC with mass analysis by light scattering [30° C., PBS (10 mM phosphate, 2.7 mM KCl, 0.137 M NaCl, pH 7.4), 0.5 mL/min, two Suprema Linear M mixed-bed columns (Polymer Standards Services, Silver Springs, Md.), do/dc 0.135 mL/g] gave M_{w} 7,100, M_{w}/Mₙ 1.2, M_{Z}/M_{W} 1.2, and IV 9.1 mL/g.

The end group conversion was determined to be 99% by acetylation of residual hydroxyl end groups and analysis by ¹H NMR as follows. A sample of 8-arm star PEG-Cl (0.2 g) was dissolved in a mixture of 0.25 mL of acetic anhydride and 0.35 mL of pyridine and left at ambient temperature overnight. The reaction was quenched by addition of 5 g of ice. The aqueous layer was extracted with three 3-mL portions of chloroform, and the combined chloroform extracts were washed successively with three 1-mL portions of 20% aqueous sodium bisulfate, two 1-mL portions of saturated aqueous sodium bicarbonate, and 1 mL of water. The chloroform was evaporated under reduced pressure. The residue was dissolved in 2 mL of water, and the resulting cloudy solution was concentrated until clear under reduced pressure to remove residual chloroform. The solution was frozen and lyophilized, and the residue was dissolved in DMSO-d₆ and analyzed by ¹H NMR.

The proportion of residual hydroxyl end groups in the 8-arm star PEG-Cl was determined by comparing the integrals of the ¹H NMR peaks for the —CH₂OAc end groups [δ 4.09 (t, J=4.7 Hz, 2H, CH₂OAc) and 2.00 (s, 3H, AcO)] with that of the CH₂Cl end groups [δ 3.72-3.68 (m, 2H, CH₂Cl)].

The 8-arm star PEG-Cl (100.9 g), was dissolved in 640 mL of concentrated aqueous ammonia (28 wt %) and heated in a sealed vessel (i.e., sealed Hastelloy® corrosion resistant alloy pressure vessel) at 60° C. for 48 h, resulting in a developed pressure of 40 psig (276 kPa). The solution was sparged for 1 to 2 h with dry nitrogen to drive off 50 to 70 g of ammonia. The solution was then passed through a column (500 mL bed volume) of strongly basic anion exchange resin (Purolite® A-860, The Purolite Co., Bala-Cynwyd, Pa.) in the hydroxide form. The eluant was collected, and three 250-mL portions of de-ionized water were passed through the column and collected. The aqueous fractions were combined, concentrated under reduced pressure (15 mm of mercury (2 kPa), bath temperature 60° C.) to about 200 g, frozen in portions and lyophilized to give 97.4 g of product (98% yield).

Treatment of the 8-arm star PEG-NH₂ with excess acetic anhydride in pyridine, as described above, and examination of the product in DMSO-d₆ by ¹H NMR indicated complete conversion of the chloride end groups and an overall 99% conversion of —OH end groups to —NH₂ end groups. The proportion of residual hydroxyl end groups in the 8-arm star PEG-NH₂ was determined by comparing the integral of the ¹H NMR peak for the —OAc end groups [δ 2.00 (s)] with that of the —NHAc end groups [δ 1.78 (s)].

### Preparation of 4-Arm Polyethylene Glycol 2K Tetraamine

A 4-arm PEG 2K tetraamine was prepared using a similar procedure as described above for the 8-arm PEG 10K octaamine.

Four-arm star PEG-OH, Mₙ 2,000 (determined by hydroxyl end group titration assuming all the polymer molecules have four arms), was obtained from NOF America (White Plains, N.Y.). The 4-arm star PEG-OH (100 g in a 500-mL round-bottom flask) was dissolved in 100 mL of dichloromethane. Thionyl chloride (88 mL, 1.2 mol) was added, and the mixture was stirred under a blanket of nitrogen at ambient temperature for 24 h. Excess thionyl chloride and dichloromethane were removed by rotary evaporation (bath temp 40° C.). Two successive 50-mL portions of toluene were added and evaporated under reduced pressure (15 mm of mercury (2 kPa), bath temperature 60° C.) to complete the removal of thionyl chloride. The yield of 4-arm star PEG-Cl was 100.1 g (97%).

¹H NMR (500 MHz, DMSO-d₆) δ 3.71-3.68 (m, 8H), 3.67-3.65 (m, 8H), 3.57-3.55 (m, 8H), 3.50 (m, ˜140H), 3.47-3.45 (m, 8H), 3.31 (s, 8H). Aqueous SEC with mass analysis by light scattering [30° C., PBS (10 mM phosphate, 2.7 mM KCl, 0.137 M NaCl, pH 7.4), 0.5 mL/min, two Polymer Standards Services Suprema Linear M mixed-bed columns, dn/dc 0.135 mL/g] gave M_{w} 1,890, M_{w}/Mₙ 1.1, M_{Z}/M_{W} 1.0, IV 5.7 mL/g.

The conversion of hydroxyl end groups to chloride end groups was determined to be 98% using the method described above for the preparation of the 8-arm PEG 10K octaamine.

The 4-arm star PEG-Cl (39.15 g) was dissolved in 600 mL of concentrated aqueous ammonia (28 wt %) and heated in a sealed vessel (i.e., sealed Hastelloy® corrosion resistant alloy pressure vessel) at 60° C. for 48 h, resulting in a developed pressure of about 40 psig (276 kPa). The solution was sparged for 1.5 h with dry nitrogen and then concentrated by rotary evaporation (15 mm of mercury (2 kPa), bath temperature 60° C.) to about 500 g. The solution was then passed through a column (500 mL bed volume) of strongly basic anion exchange resin (Purolite® A-860) in the hydroxide form. The eluant was collected, and two 250-mL portions of de-ionized water were passed through the column and collected. The aqueous fractions were combined and evaporated under reduced pressure (15 mm of mercury (2 kPa), bath temperature 60° C.) to give 36.43 g (97% yield).

¹H NMR (500 MHz, CDCl₃) δ 3.65-3.51 (m, ˜170H), 3.47 (m, 8H), 3.36 (s, 8H), 2.86 (t, J=5.3 Hz, 7.4H), 2.76 (t, J=5.4 Hz, 0.6H). Aqueous SEC with mass analysis by light scattering [30° C., PBS (10 mM phosphate, 2.7 mM KCl, 0.137 M NaCl, pH 7.4), 0.5 mL/min, two Polymer Standards Services Suprema Linear M mixed-bed columns, dn/dc 0.135 mL/g] gave M_{w} 2,330, M_{w}/Mₙ 1.2, M_{Z}/M_{w} 1.3, IV 2.2 mL/g.

Treatment of the 4-arm star PEG-NH₂ with excess acetic anhydride in pyridine and examination of the product by ¹H NMR, as described above, indicated complete conversion of the chloride end groups and an overall 96% conversion of —OH end groups to —NH₂ end groups.

### Preparation of 8-Arm Polyethylene Glycol 40K Hexadecaamine

An 8-arm PEG (Mₙ=40,000) having two amine groups on each arm was prepared using a two step procedure, as described by Arthur (copending and commonly owned Patent Application No. PCT/U.S.07/24393). An 8-arm PEG 40K polyol was reacted with thionyl chloride to produce 8-arm PEG 40K chloride, which was subsequently reacted with tris(2-aminoethyl)amine to give the 8-arm PEG 40K hexadecaamine.

### Preparation of 8-Arm PEG 40K Chloride

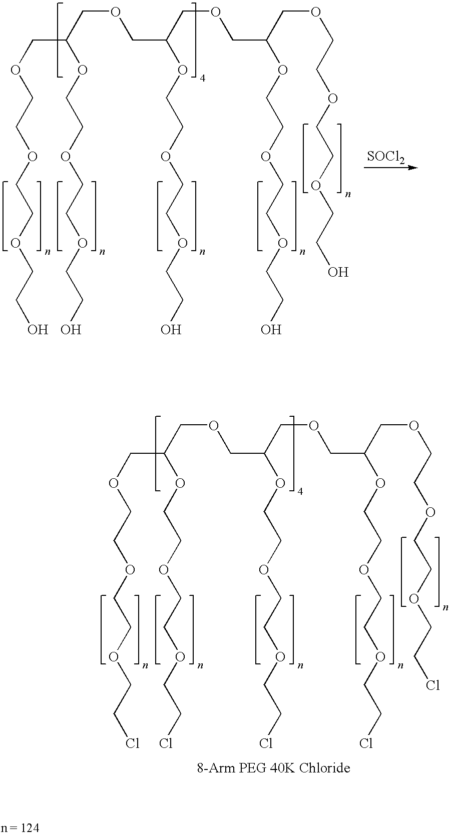

A solution of 100 g (20 mmol OH) of 8-arm PEG 40K (Mₙ=40,000; NOF SunBright HGEO-40000) in 200 mL of toluene was heated to 70° C. and stirred under nitrogen as 6 mL of thionyl chloride (10 g; 80 mmol) was quickly added. The mixture was stirred at 60° C. under nitrogen for 20 h. After 20 h the solution was bubbled with nitrogen for 1 h while still warm to remove thionyl chloride and then 2 mL (50 mmol) of methanol was added to scavenge remaining thionyl chloride. The resulting solution was added with stirring to 300 mL of hexane to initially make a gelatinous precipitate which soon became friable and powdery as the toluene extracted from the product. The white suspension was stirred for an hour and then vacuum-filtered, washed once with 100 mL of hexane and vacuum-dried under a nitrogen blanket to yield 99.0 g of 8-arm PEG 40K chloride.

### Preparation of 8-Arm PEG 40K Hexadecaamine

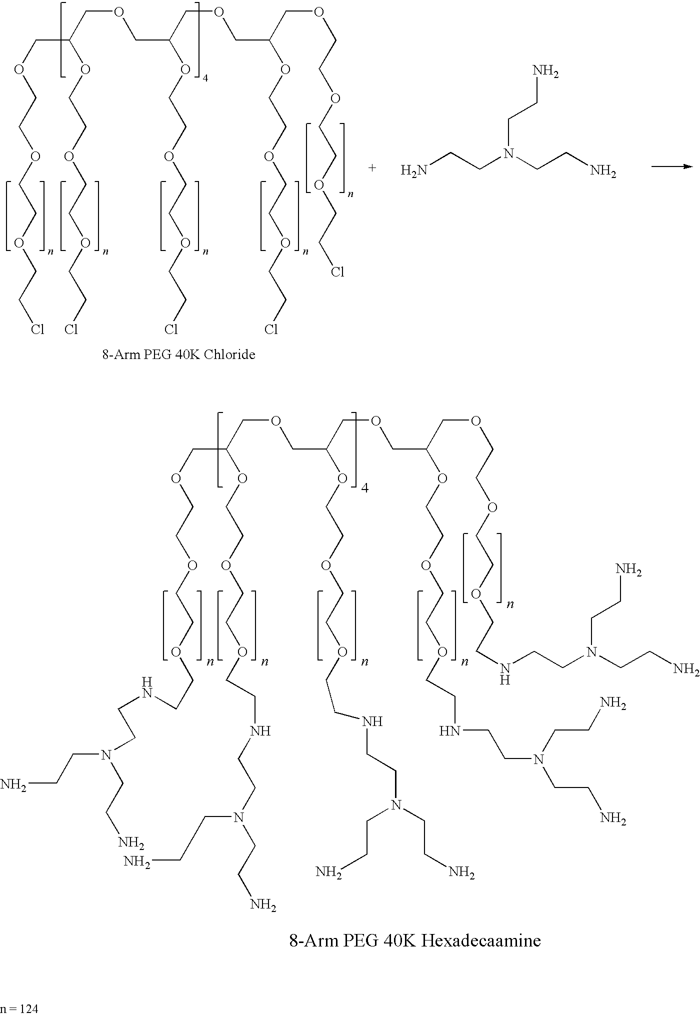

A solution of 30.0 g (6.0 mmol Cl) of 8-arm PEG 40K chloride in 60 mL of water was rapidly stirred as 36 mL (35.3 g; 240 mmol) of tris(2-aminoethyl)amine (TCI America #T1243) was added. The resulting solution was stirred in a 100° C. oil bath under nitrogen for 25 h. Then, 0.5 mL (9 mmol) of 50% sodium hydroxide was added and the mixture was cooled and extracted with 150 mL of dichloromethane followed by 2 extractions with 100 mL portions of dichloromethane. Separation was somewhat slow but eventually complete overnight. The combined extracts were dried with sodium sulfate, evaporated to a volume of 120 mL using rotary evaporation, and precipitated into 850 mL of ether with stirring. The ether was then stirred in an ice bath and the resulting white precipitate was vacuum-filtered under nitrogen, washed with 100 mL of diethyl ether and dried under nitrogen to yield 27.7 g (92% yield) of 8-arm PEG 40K hexadecaamine.

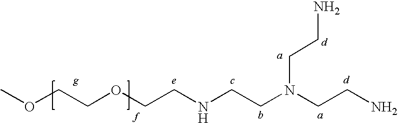

¹H NMR (CDCl₃): 2.53 ppm (t, J=6.0 Hz, a); 2.60 (t, J=6.1 Hz, b); 2.71 (t, J=6.1 Hz, c); 2.76 (t, J=5.9 Hz, d); 2.80 (t, J=5.2 Hz, e); 3.59 (t, J=5.3 Hz, f); 3.64 (s, g); 3.76 CH₂Cl (t, J=6.0 Hz; h; gone). Integrate groups of peaks: 2.5-2.8 ppm (a-e; 14.3H; theory 14H); 3.5-3.8 ppm (f-g, PEG backbone, 500H). There was no remaining tris(2-aminoethyl)amine by NMR.

Care must be taken to protect aqueous or wet organic solutions of these branched-end amines from atmospheric carbon dioxide, as carbamate formation is very facile. These carbamates will complex with divalent ions such as magnesium. When attempting to dry a dichloromethane solution of the PEG carbamate with magnesium sulfate, a clear, viscoelastic rubber was produced. The viscoelastic nature of the PEG solution in the presence of MgSO₄ is apparently due to Mg⁺² bridging the carbamate end groups.

### Example 1

### Extending Gelation Time of a Dextran Aldehyde-PEG Amine Hydrogel Using Glucosamine Free Base

The purpose of this Example was to extend the gelation time of a hydrogel formed by reacting a mixture of dextran aldehydes with a mixture of a 4-arm PEG amine and an 8-arm PEG amine using different amounts of glucosamine free base. Glucosamine was combined with an aqueous solution of dextran aldehyde in order to reduce the number of active aldehydes, resulting in a slower and more-controlled gelation time upon combination with an aqueous solution of the mixed multi-arm PEG amines.

The following aqueous solutions were prepared:

1A: a dextran aldehyde solution prepared by combining in equal volumes a 25 wt % dextran aldehyde solution (50% oxidative conversion, average molecular weight 8,500-11,500, prepared using the method described in General Methods) and a 25 wt % dextran aldehyde solution (20% oxidative conversion, average molecular weight 8,500-11,500, prepared using the method described in General Methods), mixed EW=227;

1B: a PEG amine solution prepared by combining in equal volumes a 50 wt % 4-arm PEG amine solution (Mₙ=2000, prepared as described in General Methods) and a 50 wt % 8-arm PEG amine solution (Mₙ=10,000, prepared as described in General Methods), mixed EW=670;

1C: a 19 wt % glucosamine free base solution formed by neutralizing a 25 wt % aqueous solution of glucosamine hydrochloride (obtained from Sigma-Aldrich) with one equivalent of sodium hydroxide to give a 19 wt % solution of free glucosamine containing one equivalent of sodium chloride.

Varying amounts of the glucosamine solution (1C, see Table 1) were added to 0.10 mL of the dextran aldehyde solution (1A) in a vial and the mixture was incubated for different lengths of time. Then, 0.10 mL of the PEG amine solution (1B) was added to the vial and the mixture was stirred with a small spatula until it gelled to the point where it held its shape without flowing. This time was measured and taken as the gelation time. While, the total amount of polymer solids decreased with additions of the glucosamine solution, the ratio of aldehyde groups to PEG amine groups remained constant at 1.5. The results are shown in Table 1.

TABLE 1
Effect of Glucosamine Free Base on Gelation Times of
Dextran Aldehyde-PEG Amine Hydrogels

| | | | | Gelation | Gelation |
|---|---|---|---|---|---|
| | | | | Time (sec) | Time (sec) |
| Vol of | Vol of | Vol of | | after 1 h | after 18 h |
| 1A | 1B | 1C | Glucosamine/ | incubation | incubation |
| (mL) | (mL) | (μL) | aldehyde | of 1A &1C | of 1A &1C |
| 0.1 | 0.1 | 0 | 0 | 4 | 4 |
| 0.1 | 0.1 | 8 | 0.08 | 4 | 4 |
| 0.1 | 0.1 | 15 | 0.15 | 4 | 4 |
| 0.1 | 0.1 | 23 | 0.22 | 4 | 4 |
| 0.1 | 0.1 | 31 | 0.30 | 4 | 9 |
| 0.1 | 0.1 | 39 | 0.38 | 4 | 11 |

The results demonstrate that the gelation time of the hydrogel was extended by over 100% at the highest glucosamine levels after an 18 h incubation with the dextran aldehyde solution. No effect was observed under these conditions with a shorter (1 h) incubation.

### Example 2

### Extending Gelation Time and Time-to-Tack-Free of a Dextran Aldehyde-PEG Amine Hydrogel Using Glucosamine Free Base at High pH

The purpose of this Example was to extend the gelation time and time to-tack-free of a hydrogel formed by reacting dextran aldehyde with an 8-arm PEG amine using glucosamine free base at high pH.

Dextran aldehyde, average molecular weight 8,500-11,500; 40% oxidative conversion, prepared using the method described in General Methods, (0.20 g, 1.10 mmol of aldehyde) and 0.24 g (1.10 mmol) of glucosamine hydrochloride were dissolved in 0.72 g of deionized water. After dissolution was complete, 0.088 g of a 50% sodium hydroxide solution (1.10 mmol) was added to convert the glucosamine hydrochloride to the free base form. An 8-arm PEG amine (Mₙ=10,000, Nektar) solution (30 wt %) was prepared in deionized water. The dextran aldehyde/glucosamine solution and the 8-arm PEG amine solution were mixed in ratios of 1:1, 2:1, and 1:2 and the gelation times and the time-to-tack-free were measured. A control hydrogel prepared using a dextran aldehyde solution without the added glucosamine was also tested. The control had a time-to-tack-free of about 26 sec, while the mixtures having the added glucosamine had a gelation time of about 15 min, and a time-to-tack-free between 30 and 40 min. As can be seen from these results, the glucosamine free base greatly extended both the gelation time and time-to-tack-free of the hydrogels. The effect of the glucosamine free base in this Example was larger than that observed in Example 1 because a higher ratio of glucosamine to aldehyde groups (1:1) was used. Additionally, the pH of the mixture was higher (pH of about 12) than that used in Example 1, so that a combined effect of glucosamine and high pH was observed.

### Example 3

### Extending Gelation Time of a Dextran Aldehyde-PEG Amine Hydrogel Using Glucosamine Hydrochloride

The purpose of this Example was to extend the gelation time of a hydrogel formed by reacting a mixture of dextran aldehydes with a mixture of a 4-arm PEG amine and an 8-arm PEG amine using different amounts of glucosamine hydrochloride. Glucosamine hydrochloride was combined with an aqueous solution of dextran aldehyde in order to reduce the number of active aldehydes. The lower pH resulting from the hydrochloride salt also reduced the number of active amines in the mixture resulting from the combination of the dextran aldehyde solution and the PEG amine solution. The addition of the glucosamine hydrochloride resulted in a slower and more-controlled gelation time for the hydrogel.

The following aqueous solutions were prepared:

2A: a dextran aldehyde solution prepared by combining in equal volumes a 25 wt % dextran aldehyde solution (50% oxidative conversion, average molecular weight 8,500-11,500, prepared using the method described in General Methods) and a 25 wt % dextran aldehyde solution (20% oxidative conversion, average molecular weight 60,000-90,000, prepared using the method described in General Methods), mixed EW=227;

2B: a PEG amine solution prepared by combining in equal volumes a 50 wt % 4-arm PEG amine solution (Mₙ=2000, prepared as described in General Methods) and a 50 wt % 8-arm PEG amine solution (Mₙ=10,000, prepared as described in general methods), mixed EW=670;

2C: a 25 wt % solution of glucosamine hydrochloride (glucosamine HCl).

Varying amounts of the glucosamine HCl solution (2C, see Table 2) were combined with 0.10 mL of the dextran aldehyde solution (2A) in a vial. Within 1 min of combining the glucosamine HCl and dextran aldehyde, 0.10 mL of the PEG amine solution (2B) was added and the mixture was stirred with a small spatula until it had gelled to the point that it held its shape without flowing. This time was measured and taken as the gelation time. While the total amount of polymer solids decreased with additions of the glucosamine solution, the ratio of aldehyde groups to PEG amine groups remained constant at 1.5. The results are shown in Table 2.

TABLE 2
Effect of Glucosamine HCl on Gelation Times of
Dextran Aldehyde-PEG Amine Hydrogels

| | Vol of | Vol of | Glucosamine | |
|---|---|---|---|---|
| Vol of 2A | 2B | 2C | HCl/ | Gelation |
| (mL) | (mL) | (μL) | amine | Time (sec) |
| 0.1 | 0.1 | 0 | 0 | 4 |
| 0.1 | 0.1 | 8 | 0.08 | 4 |
| 0.1 | 0.1 | 15 | 0.16 | 6 |
| 0.1 | 0.1 | 23 | 0.24 | 8 |
| 0.1 | 0.1 | 31 | 0.33 | 16 |
| 0.1 | 0.1 | 39 | 0.41 | 21 |
| 0.1 | 0.1 | 78 | 0.82 | ~30 min |

The results demonstrate that the gelation time of the hydrogel was extended by varying amounts by the addition of increasing amounts of glucosamine HCl. No incubation of the glucosamine with the dextran aldehyde was required. The extended gelation times obtained in these experiments reflect the reduction of the number of free amine groups by hydrogen ions from the hydrochloride.

### Example 4

### Decreasing Degradation Time of a Dextran Aldehyde-PEG Amine Hydrogel Using Glucosamine Hydrochloride

The purpose of this Example was to decrease the degradation time of a hydrogel formed by reacting dextran aldehyde with an 8-arm PEG amine using glucosamine hydrochloride.

An oxidized dextran solution (25 wt %, average molecular weight 8,500-11,500; 40% oxidation conversion, prepared using the method described in General Methods) and an 8-arm PEG amine solution (25 wt %) containing 2.41 wt % glucosamine hydrochloride were prepared in water. A hydrogel plug was formed by mixing the two solutions and the plug was cured for 35 min. After this time, the cured plug was placed inside a jar containing pH 7.4 phosphate buffer solution and the jar was placed inside a temperature-controlled shaker set at 80 rpm and 37° C.

After a 30 min incubation, the plug had swollen extensively and was too fragile to permit weighing. The sample disintegrated after incubation overnight. A plug treated in the same manner, but formed without the glucosamine had a degradation time of greater than two weeks.

### Example 5

### Extending Gelation Time of a Dextran Aldehyde-Jeffamine® Triamine Hydrogel Using Glucosamine Hydrochloride

The purpose of this Example was to extend the gelation time of a hydrogel formed by reacting dextran aldehyde with a Jeffamine® triamine using different amounts of glucosamine hydrochloride.

Jeffamine BA-509 (Huntsman LLC., Houston, Tex.) was dissolved in methanol to give a 75 wt % solution. A 25 wt % dextran aldehyde solution was prepared by adding the appropriate amount of oxidized dextran (average molecular weight 8,500-11,500; degree of oxidation of 50%, prepared using the method described in General Methods) to water. Then, different amounts of glucosamine hydrochloride were added to 1 mL aliquots of the dextran aldehyde solution to give the desired glucosamine concentration.

Hydrogels were formed by pipetting 24 μL of the dextran aldehyde/glucosamine solution onto a glass slide, adding 8 μL of the Jeffamine solution, and then mixing the solutions using a wooden spatula. The time to gel formation was measured. The results are given in Table 3.

TABLE 3
Gelation Time as a Function of Glucosamine HCl Concentration

| Glucosamine | | |
|---|---|---|
| Concentration | Glucosamine/NH₂ | Gelation Time |
| (wt %) | Mol Ratio | (min) |
| 0 | 0 | 5.5 |
| 0.58 | 0.036 | 4.5 |
| 1.4 | 0.087 | 2.5 |
| 2.5 | 0.155 | 3.5 |
| 3.2 | 0.201 | 5.6 |
| 4.3 | 0.270 | 9.5 |

These results demonstrate that the gelation time of a hydrogel formed by reacting dextran aldehyde with a Jeffamine® triamine can be extended by adding glucosamine, but concentrations greater than 3.2 wt % are required.

### Example 6

### Extending Gelation Time of a Dextran Aldehyde-PEG Amine Hydrogel Using D-Glucose

The purpose of this Example was to extend the gelation time of a hydrogel formed by reacting dextran aldehyde with an 8-arm PEG amine using glucose. Glucose, when combined and incubated with the PEG amine solution, reacts with the amine groups, thereby reducing the number of active amine groups available for crosslinking with the dextran aldehyde.

A 25 wt % solution of dextran aldehyde was prepared by adding the appropriate amount of oxidized dextran (average molecular weight 8,500-11,500; degree of oxidation of 50%, prepared using the method described in General Methods) to water. The PEG amine solution was prepared by dissolving 0.0231 g of 8-arm PEG amine in 690 μL of an aqueous solution containing 0.531 μmol of D-glucose. The PEG amine/glucose solution was incubated for various times (see Table 4) at room temperature. Then, 70 μL of the dextran aldehyde solution and 70 μL of the PEG amine/glucose solution were mixed and the gelation time was measured. The results are given in Table 4.

TABLE 4
Effect of Glucose on the Gelation Time of
Dextran Aldehyde-PEG Amine Hydrogels

| Incubation | |
|---|---|
| Time of | |
| Glucose | Gelation |
| and PEG | Time |
| Amine (h) | (sec) |
| 0 | 10-15 |
| 2 | 40 |
| 24 | 60 |
| 72 | 40 |
| 144 | 25-30 |

The results demonstrate that glucose extends the gelation time of a hydrogel formed by reacting dextran aldehyde and an 8-arm PEG amine when the glucose is pre-incubated with the PEG amine. No effect on gelation time was observed when the glucose was added to the dextran aldehyde solution.

### Example 7

### Extending Gelation Time of a Dextran Aldehyde-PEG Amine Hydrogel Using Hydrochloric Acid

The purpose of this Example was to extend the gelation time of a hydrogel formed by reacting dextran aldehyde with an 8-arm PEG amine using hydrochloric acid. Hydrochloric acid was combined with an aqueous solution of a multi-arm PEG amine in order to reduce the number of active amines upon combination of this mixture with an aqueous solution of dextran aldehyde. This resulted in a slower and more-controlled gelation time for the mixture.

The following solutions were used:

3A: an aqueous 25 wt % dextran aldehyde solution (48% oxidative conversion; average molecular weight 8,500-11,500, prepared using the method described in General Methods);

3B: an aqueous 20 wt % 8-arm PEG amine solution (Mₙ=10,000; Nektar); 3C, 4.0 M HCl in dioxane (Aldrich #345547).

The PEG amine solution (3B; 0.200 mL) was placed in a 3-mL vial and varying amounts of HCl (3C; see Table 5) were added with a microliter syringe. The mixture was stirred for 1 min; then 0.200 mL of dextran aldehyde solution (3A) was added to the vial and the mixture was stirred with a spatula until it gelled. The results are shown in Table 5.

TABLE 5
Effect of HCl on Gelation Time

| | | | | Gelation |
|---|---|---|---|---|
| Vol of 3A | Vol of 3B | Vol of 3C | | Time |
| (mL) | (mL) | (μL) | HCl/amine | (sec) |
| 0.2 | 0.2 | 0 | 0 | 8 |
| 0.2 | 0.2 | 1.6 | 0.20 | 10 |
| 0.2 | 0.2 | 2.8 | 0.35 | 17 |
| 0.2 | 0.2 | 3.2 | 0.40 | 19 |
| 0.2 | 0.2 | 3.6 | 0.45 | 25 |
| 0.2 | 0.2 | 4.0 | 0.50 | 35 |
| 0.2 | 0.2 | 4.4 | 0.55 | 33 |
| 0.2 | 0.2 | 4.8 | 0.60 | 40 |
| 0.2 | 0.2 | 5.2 | 0.65 | 45 |
| 0.2 | 0.2 | 6.4 | 0.80 | >5 min |
| 0.2 | 0.2 | 8.0 | 1.00 | did not gel |

The results demonstrate that the gelation time of the dextran aldehyde-PEG amine hydrogel can be extended by adding HCl to lower the pH, thereby reducing the number of active amines available for crosslinking with the dextran aldehyde.

### Example 8

### Extending Gelation Time of a Dextran Aldehyde-PEG Amine Hydrogel Using Acetic Acid

The purpose of this Example was to extend the gelation time of a hydrogel formed by reacting dextran aldehyde with an 8-arm PEG amine using acetic acid. Acetic acid was combined with an aqueous solution of a multi-arm PEG amine in order to reduce the number of active amines upon combination of this mixture with an aqueous solution of dextran aldehyde. This resulted in a slower and more-controlled gelation time for the mixture.

The following solutions were used:

4A: an aqueous 25 wt % dextran aldehyde solution (48% oxidative conversion; average molecular weight 8,500-11,500, prepared using the method described in General Methods);

4B: an aqueous 20 wt % 8-arm PEG amine solution (Mₙ=10,000; Nektar);

4C: glacial acetic acid.

The PEG amine solution (4B; 0.200 mL) was placed in a 3-mL vial and varying amounts of acetic acid (4C; see Table 6) were added with a microliter syringe. The mixture was stirred for 1 min; then 0.200 mL of dextran aldehyde solution (4A) was added to the vial and the mixture was stirred with a spatula until it gelled. The results are shown in Table 6.

TABLE 6
Effect of Acetic Acid on Gelation Time

| | | | | Gelation |
|---|---|---|---|---|
| Vol of 4A | Vol of 4B | Vol of 4C | Acetic | Time |
| (mL) | (mL) | (μL) | acid/amine | (sec) |
| 0.2 | 0.2 | 0 | 0 | 10 |
| 0.2 | 0.2 | 0.4 | 0.20 | 12 |
| 0.2 | 0.2 | 0.7 | 0.35 | 19 |
| 0.2 | 0.2 | 0.8 | 0.40 | 21 |
| 0.2 | 0.2 | 0.9 | 0.45 | 23 |
| 0.2 | 0.2 | 1.0 | 0.50 | 40 |
| 0.2 | 0.2 | 1.1 | 0.55 | 39 |
| 0.2 | 0.2 | 1.2 | 0.60 | 45 |
| 0.2 | 0.2 | 1.3 | 0.65 | 100 |
| 0.2 | 0.2 | 1.6 | 0.80 | >5 min |
| 0.2 | 0.2 | 2.0 | 1.00 | did not gel |

The results demonstrate that the gelation time of the dextran aldehyde-PEG amine hydrogel can be extended by adding acetic acid to lower the pH, thereby reducing the number of active amines available for crosslinking with the dextran aldehyde.

### Example 9

### Extending Gelation Time of a Dextran Aldehyde-PEG Amine Hydrogel Using 2-Aminoethanol

The purpose of this Example was to extend the gelation time of a hydrogel formed by reacting a mixture of dextran aldehydes with a mixture of a 4-arm PEG amine and an 8-arm PEG amine using different amounts of 2-aminoethanol. 2-Aminoethanol was combined with an aqueous solution of dextran aldehyde in order to reduce the number of active aldehydes, resulting in a slower and more-controlled gelation time upon combination with an aqueous solution of multi-arm PEG amines. The 2-aminoethanol was also combined with the PEG amine solution to act as a competitive inhibitor upon combination with dextran aldehyde solution.

The following aqueous solutions were prepared:

5A: a dextran aldehyde solution prepared by combining in equal volumes a 25 wt % dextran aldehyde solution (50% oxidative conversion, average molecular weight 8,500-11,500, prepared using the method described in General Methods) and a 25 wt % dextran aldehyde solution (20% oxidative conversion, average molecular weight 60,000-90,000, prepared using the method described in General Methods), mixed EW=227;

5B: a PEG amine solution prepared by combining in equal volumes a 50 wt % 4-arm PEG amine solution (Mₙ=2000, prepared as described in General Methods) and a 50 wt % 8-arm PEG amine solution (Mₙ=10,000, prepared as described in General Methods), mixed EW=670;

5C: a 10 wt % aqueous solution of 2-aminoethanol. Varying amounts of the 2-aminoethanol solution (5C; see Table 7) were combined with 0.10 mL of aqueous dextran aldehyde solution (5A) in a vial and incubated for 1 h, as shown in Table 7. Then, 0.10 mL of PEG amine solution (5B) was added and the mixture was stirred with a small spatula until it had gelled to the point that it held its shape without flowing. This time was measured and taken as the gelation time. While total polymer solids in the hydrogel dropped as the amount 2-aminoethanol solution was increased, the ratio of aldehyde:amine remained constant at 1.5. The results are shown in Table 7.

TABLE 7
Effect of 2-Aminoethanol on Gelation Time When
Added to the Dextran Aldehyde Solution

| Vol of 5A | Vol of 5B | Vol of 5C | Aminoethanol/ | Gelation |
|---|---|---|---|---|
| (mL) | (mL) | (μL) | aldehyde | Time (sec) |
| 0.10 | 0.10 | 0 | 0 | 4 |
| 0.10 | 0.10 | 5 | 0.07 | 4 |
| 0.10 | 0.10 | 10 | 0.15 | 7 |
| 0.10 | 0.10 | 15 | 0.22 | 13 |
| 0.10 | 0.10 | 20 | 0.30 | 22 |
| 0.10 | 0.10 | 25 | 0.37 | 46 |
| 0.10 | 0.10 | 50 | 0.75 | ~30 min |

Varying amounts of the 2-aminoethanol solution (5C; see Table 8) were also combined with 0.10 mL of the aqueous PEG amine solution (5B) in a vial and incubated for 1 min. Then, 0.10 mL of the dextran aldehyde solution was added, and the mixture was stirred with a small spatula until it had gelled. While total polymer solids in the hydrogel dropped as the amount of 2-aminoethanol solution was increased, the ratio of aldehyde:amine remained constant at 1.5. The results are given in Table 8.

TABLE 8
Effect of 2-Aminoethanol on Gelation Time When
Added to the PEG Amine Solution

| Vol of 5A | Vol of 5B | Vol of 5C | Aminoethanol/ | Gelation |
|---|---|---|---|---|
| (mL) | (mL) | (μL) | aldehyde | Time (sec) |
| 0.10 | 0.10 | 0 | 0 | 4 |
| 0.10 | 0.10 | 5 | 0.07 | 5 |
| 0.10 | 0.10 | 10 | 0.15 | 6 |
| 0.10 | 0.10 | 15 | 0.22 | 6 |
| 0.10 | 0.10 | 20 | 0.30 | 6 |
| 0.10 | 0.10 | 25 | 0.37 | 6 |
| 0.10 | 0.10 | 50 | 0.75 | 10 |

The results demonstrate that the gelation time of the dextran aldehyde-PEG amine hydrogel can be extended by adding 2-aminoethanol to either the dextran aldehyde solution or the PEG amine solution, although a larger effect is obtained with addition to the dextran aldehyde solution.

### Example 10

### Extending Gelation Time of a Dextran Aldehyde-PEG Amine Hydrogel Using 2-Aminoethanol Hydrochloride

The purpose of this Example was to extend the gelation time of a hydrogel formed by reacting a mixture of dextran aldehydes with a mixture of a 4-arm PEG amine and an 8-arm PEG amine using different amounts of 2-aminoethanol hydrochloride. 2-Aminoethanol hydrochloride was combined with an aqueous solution of PEG amine in order to reduce the number of active amines, resulting in a slower and more-controlled gelation time upon combination with an aqueous solution of dextran aldehyde.

The following aqueous solutions were prepared:

6A: a dextran aldehyde solution prepared by combining in equal volumes a 25 wt % dextran aldehyde solution (50% oxidative conversion, average molecular weight 8,500-11,500, prepared using the method described in General Methods) and a 25 wt % dextran aldehyde solution (20% oxidative conversion, average molecular weight 60,000-90,000, prepared using the method described in General Methods), mixed EW=227;

6B: a PEG amine solution prepared by combining in equal volumes a 50 wt % 4-arm PEG amine solution (Mₙ=2000, prepared as described in General methods) and a 50 wt % 8-arm PEG amine solution (Mₙ=10,000, prepared as described in General Methods), mixed EW=670;

6C: a 16 wt % aqueous solution of 2-aminoethanol hydrochloride, prepared by adding 1.35 mL of concentrated HCL (12.1 M) to 1.00 g of 2-aminoethanol in 5 g of water and then adding water to give a total weight of 10.0 g of solution.

Varying amounts of the 2-aminoethanol HCl solution (6C; see Table 9) were combined with 0.10 mL of aqueous PEG amine solution (6B) in a vial.

Within 1 min of combining the 2-aminoethanol HCl and PEG amine solutions, 0.10 mL of dextran aldehyde solution (6A) was added and the mixture was stirred with a small spatula until it had gelled to the point that it held its shape without flowing. This time was measured and taken as the gelation time. While total polymer solids in the hydrogel dropped as the amount of 2-aminoethanol HCl solution was increased, the ratio of aldehyde:amine remained constant at 1.5. The results are shown in Table 9.

TABLE 9
Effect of 2-Aminoethanol HCl on Gelation Time When
Added to the PEG Amine Solution

| Vol of 6A | Vol of 6B | Vol of 6C | | Gelation |
|---|---|---|---|---|
| (mL) | (mL) | (μL) | HCl/amine | Time (sec) |
| 0.10 | 0.10 | 0 | 0 | 4 |
| 0.10 | 0.10 | 5 | 0.11 | 5 |
| 0.10 | 0.10 | 10 | 0.22 | 6 |
| 0.10 | 0.10 | 15 | 0.33 | 9 |
| 0.10 | 0.10 | 20 | 0.43 | 12 |
| 0.10 | 0.10 | 25 | 0.54 | 15 |
| 0.10 | 0.10 | 50 | 1.09 | 30 |

The results demonstrate that the gelation time of the dextran aldehyde-PEG amine hydrogel can be extended by adding 2-aminoethanol hydrochloride to the PEG amine solution.

### Example 11

### Extending Gelation Time of a Dextran Aldehyde-PEG Amine Hydrogel Using Diisopropylamine

The purpose of this Example was to extend the gelation time of a hydrogel formed by reacting dextran aldehyde with an 8-arm PEG amine using different amounts of diisopropylamine. A secondary amine should bind reversibly to the aldehyde groups in dextran aldehyde and be capable of displacement by a primary amine which can form a stable imine bond. Diisopropylamine, which is a sterically-hindered secondary amine, was used to extend the gel time in a dextran aldehyde-multi-arm PEG amine hydrogel.

The following solutions were used:

7A: a 20 wt % aqueous dextran aldehyde solution (25% oxidative conversion, average molecular weight 60,000-90,000, prepared using the method described in General Methods; EW=308);

7B: a 50 wt % 8-arm PEG amine solution (Mₙ=10,000, prepared as described in General Methods; EW=1250);

7C: neat diisopropylamine (DIPA) (EW=101, D=0.72 g/mL).

Dextran aldehyde solution (7A; 0.10 mL) was placed in a 3-mL vial. Then 0.10 mL PEG amine solution (7B) was added and the mixture was stirred vigorously with a small spatula until it had gelled to the point that it held its shape without flowing. This time was measured and taken as the gelation time. Diisopropylamine was added to either one or the other of the two solutions before they were mixed to determine its effect on the gelation time. When diisopropylamine was added to the dextran aldehyde solution, it was allowed to stand for an hour at room temperature before determining the gelation time. When diisopropylamine was added to the PEG amine, the solution was used immediately. The results are given in Table 10.

TABLE 10
Effect of Diisopropylamine on Gelation Time

| | | | | Gelation | Gelation |
|---|---|---|---|---|---|
| | | | | Time | Time |
| | | | | (sec) | (sec) |
| Vol of 7A | Vol of 7B | Vol of 7C | DIPA: | DIPA in | DIPA in |
| (mL) | (mL) | (μL) | aldehyde | 7B | 7A |
| 0.10 | 0.10 | 0 | 0 | 4 | 4 |
| 0.10 | 0.10 | 2 | 0.23 | nd* | 12 |
| 0.10 | 0.10 | 4 | 0.47 | nd | 45 |
| 0.10 | 0.10 | 6 | 0.70 | 4 | 160 |
| 0.10 | 0.10 | 11 | 1.28 | 3 | 510 |
| 0.10 | 0.10 | 17 | 1.98 | 3 | nd |
| 0.10 | 0.10 | 22 | 2.56 | 3 | nd |

*“nd” means not determined.

The results demonstrate that the diisopropylamine had no effect on the gelation time of the dextran aldehyde-PEG amine hydrogel when it was added to the PEG amine solution. This result demonstrates that the hindered secondary diisopropylamines compete unfavorably with the primary PEG amines in reacting with the dextran aldehyde groups. Additionally, the resulting hydrogels were gummy. In contrast, addition of diisopropylamine to the dextran aldehyde solution resulted in an extension of the gelation time of the hydrogel. In this case the diisopropylamine was allowed enough time (1 h) to react with dextran aldehyde groups in the absence of any competing primary amines. The resulting hydrogels were soft and elastic. The dextran aldehyde solution containing the diisopropylamine became yellow-orange in color upon standing.

### Example 12

### Extending Gelation Time of a Dextran Aldehyde-PEG Amine Hydrogel Using a Peptide

The purpose of this Example was to extend the gelation time of a hydrogel formed by reacting dextran aldehyde with 8-arm PEG amine using a peptide.

The peptide used had the following amino acid sequence RTNAADHPAAVTGGGC (MW=1497 Da), given as SEQ ID NO:1, and was obtained from SynPep (Dublin, Calif.). An aqueous solution was prepared by adding the 8-arm PEG amine, in an amount sufficient to give a concentration of 23 wt %, and the peptide (two different amounts as shown in Table 11) to water. The pH of this solution was measured using pH paper to be between 9 and 10. The solution of the dextran aldehyde was prepared by adding the appropriate amount of oxidized dextran (M_{w}=10,000, degree of oxidation of 50%) to water to give a 25 wt % solution.

The two solutions were mixed by taking 15 μL of each solution and mixing manually. The gelation time and the time-to-tack-free were measured and the results are presented in Table 11.

TABLE 11
Gelation Time and Time-to-Tack-Free as
a Function of Peptide Concentration

| Peptide | Gelation | Time-to- |
|---|---|---|
| Concentration | Time | Tack-Free |
| (wt %) | (min) | (min) |
| 0 | 0.5 | 0.75 |
| 1.5 | 0.5 | 0.75 |
| 8.2 | 4 | 6 |

These results demonstrate that the gelation time and time-to-tack-free of a hydrogel formed by reacting dextran aldehyde with 8-arm PEG amine can be extended using a peptide.

### Example 13

### Extending Gelation Time of a Dextran Aldehyde-PEG Amine Hydrogel Using the Amino Acid L-Cysteine

The purpose of this Example was to extend the gelation time of a hydrogel formed by reacting a dextran aldehyde with a mixture of 8-Arm PEG 40K hexadecaamine and 4-arm PEG 2K amine using different amounts of the amino acid L-cysteine. The L-cysteine was combined with an aqueous solution of dextran aldehyde and incubated for either 20 min or 2 h before preparing the hydrogel.

An aqueous 25 wt % dextran aldehyde solution was prepared by adding 5.0 g of dextran aldehyde (average molecular weight 8,500-11,500; oxidation conversion of 50%, prepared using the method described in General Methods) to 15.0 g of doubly-distilled water and shaking the mixture overnight at 105 rpm and 37° C. to obtain a uniform solution.

A PEG amine solution (60 wt % solids) was prepared by combining 1.2 g of 8-Arm PEG 40K hexadecaamine (prepared as described in General Methods) with 4.8 g of 4-arm PEG 2K (prepared as described in General Methods) and 4.0 g of doubly-distilled water. The mixture was shaken overnight at 105 rpm and 37° C. to obtain a uniform solution.

The desired amount of L-cysteine was weighed into a vial (see Table 12).

To this solid, 100 μL of the dextran aldehyde solution was added. The mixture was shaken at 215 rpm for either 20 min or 2 h. When the amount to be added was 5.0 mg or less, both the volume and the mass added were doubled, to allow for greater accuracy when weighing.

The vial containing the solution of dextran aldehyde and cysteine was tilted and 100 μL of the PEG amine solution was added with care so that the two solutions were not mixed. A timer was started and the two solutions were stirred together with a wooden end of a cotton swab. The gelation time was defined as the time when stirring pulled the gel from the sides of the vial so that the gel could be removed as the wooden stirring rod was removed from the vial. The results are given in Table 12.

TABLE 12
Gelation Time as a Function of L-Cysteine
Concentration and Incubation Time

| | | Incubation | Gelation |
|---|---|---|---|
| L-cysteine | Cysteine/ | Time | Time |
| (mg) | aldehyde | (min) | (sec) |
| 0 | 0 | 20 | 7 |
| 2.0 | 0.09 | 20 | 8 |
| 5.0 | 0.21 | 20 | 9 |
| 7.5 | 0.32 | 20 | 10 |
| 10.0 | 0.43 | 20 | 12 |
| 0 | 0 | 120 | 7 |
| 2.0 | 0.09 | 120 | 15 |
| 5.0 | 0.21 | 120 | 19 |
| 7.5 | 0.32 | 120 | 19 |
| 10.0 | 0.43 | 120 | 23 |

These results demonstrate that the gelation time of a hydrogel formed by reacting dextran aldehyde with a mixture of PEG amines can be extended using L-cysteine. The gelation time increased with increasing amounts of L-cysteine and with a longer incubation time of L-cysteine with the dextran aldehyde.

### Example 14

### Extending Gelation Time of a Dextran Aldehyde-PEG Amine Hydrogel Using the Amino Acid L-Serine

The purpose of this Example was to extend the gelation time of a hydrogel formed by reacting a dextran aldehyde with a mixture of 8-Arm PEG 40K hexadecaamine and 4-arm PEG 2K amine using different amounts of the amino acid L-serine. The L-serine was combined with an aqueous solution of dextran aldehyde and incubated for either 20 min or 2 h before preparing the hydrogel.

The experimental procedure used was the same as that described in Example 13. The gelation times are given in Table 13.

TABLE 13
Gelation Time as a Function of L-Serine
Concentration and Incubation Time

| | | Incubation | Gelation |
|---|---|---|---|
| L-serine | Serine/ | Time | Time |
| (mg) | aldehyde | (min) | (sec) |
| 0 | 0 | 20 | 7 |
| 2.0 | 0.10 | 20 | 8 |
| 5.0 | 0.25 | 20 | 9 |
| 7.5 | 0.37 | 20 | 11 |
| 10.0 | 0.49 | 20 | 17 |
| 0 | 0 | 120 | 7 |
| 2.0 | 0.10 | 120 | 11 |
| 5.0 | 0.25 | 120 | 14 |
| 7.5 | 0.37 | 120 | 13 |
| 10.0 | 0.49 | 120 | 17 |

These results demonstrate that the gelation time of a hydrogel formed by reacting dextran aldehyde with a mixture of PEG amines can be extended using L-serine. The gelation time increased with increasing amounts of L-serine, but not with a longer incubation time of L-serine with the dextran aldehyde.

### Example 15

### Extending Gelation Time of a Dextran Aldehyde-PEG Amine Hydrogel

### Using the Amino Acid L-Lysine

The purpose of this Example was to extend the gelation time of a hydrogel formed by reacting a dextran aldehyde with a mixture of 8-Arm PEG 40K hexadecaamine and 4-arm PEG 2K amine using different amounts of the amino acid L-lysine. The L-lysine was combined with an aqueous solution of dextran aldehyde and incubated for 1 h before preparing the hydrogel.

A PEG amine solution was prepared by adding 0.30 g of 8-Arm PEG 40K hexadecaamine (prepared as described in General Methods), 0.30 g of 4-arm PEG 2K (prepared as described in General Methods) and 0.40 g water to a vial. The resulting mixture was agitated at 37° C. until a homogeneous solution was achieved.

A dextran aldehyde solution was prepared by adding to a second vial, 0.25 g of dextran aldehyde (average molecular weight 8,500-11,500, oxidation conversion of 53.7%, prepared using the method described in General Methods) and 0.75 g of water. This mixture was agitated at 37° C. until a homogeneous solution was achieved. At this point a varying quantity of L-lysine was added to the dextran aldehyde solution and the resulting mixture was agitated at room temperature for 1 h.

Then, a double-barreled syringe was assembled with plungers and the PEG amine solution was added to one of the barrels and the dextran aldehyde solution containing L-lysine was added to the remaining empty barrel. A twelve-step mixing tip was then placed onto the syringe and the plungers of the syringe were moved to expel each of the solutions onto a glass plate. The time until the gel became too stiff to be pulled up with a small wooden rod was recorded in seconds and taken as the gelation time. A number of individual samples were run in this manner with various amounts of L-lysine, and the results are shown in Table 14.

TABLE 14
Gelation Time as a Function of L-Lysine Concentration

| | | Incubation | Gelation |
|---|---|---|---|
| L-Lysine | mol % | Time | Time |
| (g) | L-Lysine* | (h) | (sec) |
| 0 | 0 | 1 | 1.5 |
| 0.03 | 11 | 1 | 3 |
| 0.033 | 12 | 1 | 3 |
| 0.035 | 13 | 1 | 4 |
| 0.038 | 14 | 1 | 4 |
| 0.054 | 20 | 1 | 8 |
| 0.062 | 23 | 1 | 45 |
| 0.068 | 25 | 1 | 35 |
| 0.081 | 30 | 1 | 67 |
| 0.108 | 40 | 1 | 110 |
| 0.136 | 50 | 1 | 480 |

*Note that the mol % of L-lysine refers to the percent of the total equivalents of potentially available aldehyde groups involved in potential reaction with the corresponding equivalents of L-lysine. It is assumed for these calculations that only one amine from L-lysine participates in reaction with the aldehyde groups.

These results demonstrate that the gelation time of a hydrogel formed by reacting dextran aldehyde with a mixture of PEG amines can be extended using L-lysine.

What is claimed is:

1. A method for extending the gelation time for at least one oxidized polysaccharide (component A) and at least one water-dispersible, multi-arm amine (component B) to form a hydrogel in an aqueous medium, said at least one oxidized polysaccharide containing aldehyde groups, having a weight-average molecular weight of about 1,000 to about 1,000,000 Daltons and an equivalent weight per aldehyde group of about 90 to about 1500 Daltons, and said at least one water-dispersible, multi-arm amine having at least three of its arms terminated by a primary amine group, and a number-average molecular weight of about 450 to about 200,000 Daltons; said method comprising:
contacting component A and component B in the presence of an aqueous medium and at least one chemical additive to form a mixture that forms a resulting hydrogel, wherein said chemical additive is biocompatible, has a molecular weight of less than about 2,000 Daltons and comprises at least one reactive group capable of reacting with amine or aldehyde groups, said reactive group being selected from the group consisting of aldehyde, ketone, glyoxal, acetoacetate, activated ester, imidoester, maleimide, p-nitrophenyl ester, activated halide, anhydride, carbonyl imidazole, epoxide, alkylhalide, H⁺, OH⁻, primary amine, secondary amine, and carboxyhydrazide, provided that the chemical additive does not induce gelation when mixed in the aqueous medium with either component (A) alone or component (B) alone;
wherein, in said method, the additive is used in an amount sufficient to extend the gelation time of components (A) and (B) under predetermined conditions by at least about 10% compared to that of said components (A) and (B) under said conditions, but in the absence of said additive.

2. The method according to claim 1 wherein the contacting is on an anatomical site on tissue of a living organism to form the mixture and the resulting hydrogel directly thereon.

3. The method according to claim 1 further comprising applying the mixture directly on an anatomical site on tissue of a living organism to form the resulting hydrogel thereon.

4. The method according to claim 1 wherein
a) the at least one oxidized polysaccharide is provided in a first aqueous solution or dispersion, said solution or dispersion containing from about 5% to about 40% by weight of the oxidized polysaccharide;
b) the at least one multi-arm amine is provided in a second aqueous solution or dispersion, said solution or dispersion containing from about 5% to about 70% by weight of the multi-arm amine, and
c) the at least one chemical additive is provided in at least one of the following:
(i) the first aqueous solution or dispersion;
(ii) the second aqueous solution or dispersion; or
(iii) a third aqueous solution or dispersion.

5. The method according to claim 4 wherein the at least one chemical additive is provided in the second aqueous solution or dispersion and comprises at least one reactive group selected from the group consisting of aldehyde, ketone, glyoxal, acetoacetate, activated ester, imidoester, maleimide, p-nitrophenyl ester, activated halide, anhydride, carbonyl imidazole, epoxide, alkylhalide, and H⁺.

6. The method according to claim 4 wherein the at least one chemical additive is provided in the first aqueous solution or dispersion and comprises at least one reactive group selected from the group consisting of primary amine, secondary amine, carboxyhydrazide, and OH⁻.

7. The method according to claim 4 wherein the concentration of the at least one oxidized polysaccharide in the first aqueous solution or dispersion is from about 5% to about 30% by weight.

8. The method according to claim 4 wherein the concentration of the at least one multi-arm amine in the second aqueous solution or dispersion is from about 20% to about 50% by weight.

9. The method according to claim 4 wherein the second aqueous solution or dispersion further comprises at least one multi-functional amine having one or more primary amine groups, said multi-functional amine being present at a concentration of about 5% to about 1000% by weight relative to the amount of the multi-arm amine in the solution.

10. The method according to claim 9 wherein the at least one multi-functional amine is selected from the group consisting of water-dispersible multi-arm polyether amines, linear and branched diamines, linear branched end amines, branched polyamines, cyclic diamines, aminoalkyltrialkoxysilanes, aminoalkyldialkoxyalkylsilanes, dihydrazides, linear polymeric diamines, comb polyamines, dihydrazides, and polyhydrazides.

11. The method according to claim 1 wherein the at least one chemical additive is selected from the group consisting of primary amines, secondary amines, aldose sugars, ketose sugars, Brønsted acids, acid salts, Brønsted bases, amino acids, peptides having between 2 and about 15 amino acids, activated esters, and activated halides.

12. The method according to claim 11 wherein the at least one chemical additive is selected from the group consisting of glucosamine, 2-aminoethanol, diisopropylamine, D-glucose, hydrochloric acid, acetic acid, glucosamine hydrochloride, 2-aminoethanol hydrochloride, sodium hydroxide, lysine, cysteine, serine, and a peptide having a sequence as set forth in SEQ ID NO:1.

13. The method according to claim 1, wherein the weight-average molecular weight of the at least one oxidized polysaccharide is from about 3,000 to about 250,000 Daltons.

14. The method according to claim 1 wherein the number-average molecular weight of the at least one multi-arm amine is from about 2,000 to about 40,000 Daltons.

15. The method according to claim 1 wherein the at least one oxidized polysaccharide is selected from the group consisting of dextran, starch, agar, cellulose, hydroxyethylcellulose, pullulan, and hyaluronic acid.

16. The method according to claim 15 wherein the at least one oxidized polysaccharide is dextran.

17. The method according to claim 1 wherein the at least one water-dispersible multi-arm amine is selected from the group consisting of amino-terminated star polyethylene oxides, amino-terminated dendritic polyethylene oxides, amino-terminated comb polyethylene oxides, amino-terminated star polypropylene oxides, amino-terminated dendritic polypropylene oxides, amino-terminated comb polypropylene oxides, amino-terminated star polyethylene oxide-polypropylene oxide copolymers, amino-terminated dendritic polyethylene oxide-polypropylene oxide copolymers, amino-terminated comb polyethylene oxide-polypropylene oxide copolymers, amino-terminated dendritic polyamidoamines, polyoxyalkylene triamines, and multi-arm branched end amines.

18. A method for decreasing the degradation time of a hydrogel formed from at least one oxidized polysaccharide (component A) and at least one water-dispersible, multi-arm amine (component B) in an aqueous medium, said at least one oxidized polysaccharide containing aldehyde groups, having a weight-average molecular weight of about 1,000 to about 1,000,000 Daltons and an equivalent weight per aldehyde group of about 90 to about 1500 Daltons, and said at least one water-dispersible, multi-arm amine having at least three of its arms terminated by a primary amine group, and a number-average molecular weight of about 450 to about 200,000 Daltons; said method comprising:
contacting component A and component B in the presence of an aqueous medium and at least one chemical additive to form a mixture that forms a resulting hydrogel, wherein said chemical additive is biocompatible, has a molecular weight of less than about 2,000 Daltons and comprises at least one reactive group capable of reacting with amine or aldehyde groups, said reactive group being selected from the group consisting of aldehyde, ketone, glyoxal, acetoacetate, activated ester, imidoester, maleimide, p-nitrophenyl ester, activated halide, anhydride, carbonyl imidazole, epoxide, alkylhalide, H⁺, OH⁻, primary amine, secondary amine, and carboxyhydrazide, provided that the chemical additive does not induce gelation when mixed in the aqueous medium with either component (A) alone or component (B) alone;
wherein, in said method, the additive is used in an amount sufficient to decrease the degradation time of the resulting hydrogel under predetermined conditions by at least about 10% compared to that of the hydrogel formed under said conditions, but in the absence of said additive.

19. The method according to claim 18 wherein the contacting is on an anatomical site on tissue of a living organism to form the mixture and the resulting hydrogel directly thereon.

20. The method according to claim 18, further comprising applying the mixture directly on an anatomical site on tissue of a living organism to form the resulting hydrogel thereon.

21. The method according to claim 18 wherein
a) the at least one oxidized polysaccharide is provided in a first aqueous solution or dispersion, said solution or dispersion containing from about 5% to about 40% by weight of the oxidized polysaccharide;
b) the at least one multi-arm amine is provided in a second aqueous solution or dispersion, said solution or dispersion containing from about 5% to about 70% by weight of the multi-arm amine, and
c) the at least one chemical additive is provided in at least one of the following:
(i) the first aqueous solution or dispersion;
(ii) the second aqueous solution or dispersion; or
(iii) a third aqueous solution or dispersion

22. The method according to claim 21 wherein the at least one chemical additive is provided in the second aqueous solution or dispersion and comprises at least one reactive group selected from the group consisting of aldehyde, ketone, glyoxal, acetoacetate, activated ester, imidoester, maleimide, p-nitrophenyl ester, activated halide, anhydride, carbonyl imidazole, epoxide, alkylhalide, and H⁺.

23. The method according to claim 21 wherein the at least one chemical additive is provided in the first aqueous solution or dispersion and comprises at least one reactive group selected from the group consisting of primary amine, secondary amine, carboxyhydrazide, and OH⁻.

24. The method according to claim 21 wherein the concentration of the at least one oxidized polysaccharide in the first aqueous solution is from about 5% to about 30% by weight.

25. The method according to claim 21 wherein the concentration of the at least one multi-arm amine in the second aqueous solution is from about 20% to about 50% by weight.

26. The method according to claim 21 wherein the second aqueous solution further comprises at least one multi-functional amine having one or more primary amine groups, said multi-functional amine being present at a concentration of about 5% to about 1000% by weight relative to the amount of the multi-arm amine in the solution.

27. The method according to claim 26 wherein the at least one multi-functional amine is selected from the group consisting of water-dispersible multi-arm polyether amines, linear and branched diamines, linear branched end amines, branched polyamines, cyclic diamines, aminoalkyltrialkoxysilanes, aminoalkyldialkoxyalkylsilanes, dihydrazides, linear polymeric diamines, comb polyamines, dihydrazides, and polyhydrazides.

28. The method according to claim 18 wherein the at least one chemical additive is selected from the group consisting of primary amines, secondary amines, aldose sugars, ketose sugars, Brønsted acids, acid salts, Brønsted bases, amino acids, peptides having between 2 and about 15 amino acids, activated esters, and activated halides.

29. The method according to claim 28 wherein the at least one chemical additive is selected from the group consisting of glucosamine, 2-aminoethanol, diisopropylamine, D-glucose, hydrochloric acid, acetic acid, glucosamine hydrochloride, 2-aminoethanol hydrochloride, sodium hydroxide, lysine, cysteine, serine, and a peptide having a sequence as set forth in SEQ ID NO:1.

30. The method according to claim 18, wherein the weight-average molecular weight of the at least one oxidized polysaccharide is from about 3,000 to about 250,000 Daltons.

31. The method according to claim 18 wherein the number-average molecular weight of the at least one multi-arm amine is from about 2,000 to about 40,000 Daltons.

32. The method according to claim 18 wherein the at least one oxidized polysaccharide is selected from the group consisting of dextran, starch, agar, cellulose, hydroxyethylcellulose, pullulan, and hyaluronic acid.

33. The method according to claim 32 wherein the at least one oxidized polysaccharide is dextran.

34. The method according to claim 18 wherein the at least one water-dispersible multi-arm amine is selected from the group consisting of amino-terminated star polyethylene oxides, amino-terminated dendritic polyethylene oxides, amino-terminated comb polyethylene oxides, amino-terminated star polypropylene oxides, amino-terminated dendritic polypropylene oxides, amino-terminated comb polypropylene oxides, amino-terminated star polyethylene oxide-polypropylene oxide copolymers, amino-terminated dendritic polyethylene oxide-polypropylene oxide copolymers, amino-terminated comb polyethylene oxide-polypropylene oxide copolymers, amino-terminated dendritic polyamidoamines, polyoxyalkylene triamines, and multi-arm branched end amines.

35. A method for forming a hydrogel on an anatomical site on tissue of a living organism by either
(a) mixing on said anatomical site in the presence of an aqueous medium at least one oxidized polysaccharide containing aldehyde groups, having a weight-average molecular weight of about 1,000 to about 1,000,000 Daltons and an equivalent weight per aldehyde group of about 90 to about 1500 Daltons, and at least one water-dispersible, multi-arm amine wherein at least three of its arms are terminated by a primary amine group, wherein the multi-arm amine has a number-average molecular weight of about 450 to about 200,000 Daltons, to form a mixture that forms a hydrogel with a determinable gelation time and a determinable degradation time, or
(b) mixing said at least one oxidized polysaccharide and said at least one multi-arm amine in the presence of an aqueous medium to form said mixture and applying said mixture to said anatomical site to form said hydrogel thereon with said determinable gelation time and said determinable degradation time, the improvement comprising the step of:
including in said mixture at least one chemical additive, wherein said chemical additive is biocompatible, has a molecular weight of less than about 2,000 Daltons and comprises at least one reactive group capable of reacting with amine or aldehyde groups selected from the group consisting of aldehyde, ketone, glyoxal, acetoacetate, activated ester, imidoester, maleimide, p-nitrophenyl ester, activated halide, anhydride, carbonyl imidazole, epoxide, alkylhalide, H⁺, OH⁻, primary amine, secondary amine, and carboxyhydrazide, provided that the chemical additive does not induce gelation when mixed in the aqueous medium with either said at least one oxidized polysaccharide alone or said at least one multi-arm amine alone, whereby the resulting mixture forms a resulting hydrogel,
wherein said additive is used in an amount sufficient to (i) increase the determinable gelation time by at least about 10%; (ii) decrease the determinable degradation time by at least about 10%; or (iii) both (i) and (ii).


SEQUENCE LISTING (1 sequence)

SEQ ID NO: 1 - protein, 16 aa, Artificial Sequence
  Synthetic Peptide
RTNAADHPAAVTGGGC